SEQUENCE LISTING (3 sequences)

SEQ ID NO: 1 - protein, 288 aa, Homo sapiens
MSDIEEVVEEYEEEEQEEAAVEEQEEAAEEDAEAEAETEETRAEEDEEEEEAKEAEDGPMEESKPKPRSFMPNLVPPKIPDGERVDFDDIHRKRMEKDLNELQALIEAHFENRKKEEEELVSLKDRIERRRAERAEQQRIRNEREKERQNRLAEERARREEEENRRKAEDEARKKKALSNMMHFGGYIQKQAQTERKSGKRQTEREKKKKILAERRKVLAIDHLNEDQLREKAKELWQSIYNLEAEKFDLQEKFKQQKYEINVLRNRINDNQKVSKTRGKAKVTGRWK

SEQ ID NO: 2 - protein, 210 aa, Homo sapiens
MADGSSDAAREPRPAPAPIRRRSSNYRAYATEPHAKKKSKISASRKLQLKTLLLQIAKQELEREAEERRGEKGRALSTRCQPLELAGLGFAELQDLCRQLHARVDKVDEERYDIEAKVTKNITEIADLTQKIFDLRGKFKRPTLRRVRISADAMMQALLGARAKESLDLRAHLKQVKKEDTEKENREVGDWRKNIDALSGMEGRKKKFES

SEQ ID NO: 3 - protein, 161 aa, Homo sapiens
MDDIYKAAVEQLTEEQKNEFKAAFDIFVLGAEDGCISTKELGKVMRMLGQNPTPEELQEMIDEVDEDGSGTVDFDEFLVMMVRCMKDDSKGKSEEELSDLFRMFDKNADGYIDLDELKIMLQATGETITEDDIEELMKDGDKNNDGRIDYDEFLEFMKGVE

(19) United States
(12) Patent Application Publication
(10) Pub. No.: US 2025/0208144 A1
(43) Pub. Date: Jun. 26, 2025

(54) ASSAY FOR LONG FORMS OF CARDIAC TROPONIN T

(71) Applicant: Turun yliopisto, Turun yliopisto (FI)

(72) Inventors: Saara WITTFOOTH, Turun yliopisto (FI); Juhani AIRAKSINEN, Turun yliopisto (FI); Tuija VASANKARI, Turun yliopisto (FI); Rami AALTO, Turun yliopisto (FI); Akseli LAHTINEN, Turun yliopisto (FI); Tapio HELLMAN, Turun yliopisto (FI)

(21) Appl. No.: 18/846,688

(22) PCT Filed: Mar. 29, 2023
(86) PCT No.: PCT/FI2023/050176

§ 371 Date: Sep. 13, 2024

(30) Foreign Application Priority Data
Mar. 29, 2022 (FI) 20225269

(51) Int. Cl. G01N 33/68; G01N 33/543

(52) U.S. Cl. CPC G01N 33/6893; G01N 33/543; G01N 33/6887; G01N 2333/4712; G01N 2800/324

(57) ABSTRACT

The invention relates to in vitro methods of determining likelihood of acute myocardial infarction in a subject on the basis of certain long forms of cardiac troponin T. The invention also relates to a kit for use in the methods.

### FIELD OF THE INVENTION

The invention relates to an assay method of determining likelihood of acute myocardial infarction (MI) in a subject and to a kit for use in the assay method.

### BACKGROUND OF THE INVENTION

High-sensitivity cardiac troponin assays are a key element in the diagnostic workup of acute coronary syndromes. Liberal use of troponin measurements has led to a situation where minor troponin elevations are a common finding in every day practice and often not caused by Type 1 myocardial infarction (MI). Other conditions such as chronic kidney disease, atrial fibrillation, strenuous exercise and heart failure may cause transient or sustained increase in plasma troponin levels, thereby causing diagnostic challenges and leading to redundant use of diagnostic coronary angiography or “overdiagnosis” of MI.

During MI cardiac troponins are released from the ischemic myocardium as a combination of full-size troponin and troponin fragments followed by proteolytic degradation in serum in a time-dependent pattern. Determinants and mechanisms of cardiac troponin release in other clinical circumstances are not fully understood. The free fragmented troponins in cytoplasm may traverse across cell membranes that have become leaky but not irreversibly damaged. Commercial high-sensitivity troponin T (cTnT) tests detect also these small fragments and may thus lead to false diagnosis of MI. There is thus an identified need for accurate diagnostic troponin tests, especially for patient groups in which elevated troponin levels are common.

### SUMMARY

In one aspect, the invention relates to an in vitro assay method of determining likelihood of acute myocardial infarction (MI) in a subject according to claim 1.

In another aspect, the invention relates to a kit for use in determining the likelihood of acute MI in a subject according to claim 17.

In some further aspects, the invention relates to a use of the kit for determining the likelihood of acute MI in a subject as set forth claim 24, and to a use of the kit for distinguishing patients having MI from patients showing elevated standard cTnT levels for a reason other than MI as set forth in claim 25.

Further aspects, embodiments and details are set forth in following figures, detailed description, examples, and dependent claims.

### BRIEF DESCRIPTION OF THE DRAWINGS

The accompanying drawings, which are included to provide a further understanding of the invention and constitute a part of this specification, illustrate embodiments of the invention and together with the description help to explain the principles of the invention. In the drawings:

FIG. 1 is a schematic representation of the molecular forms of cTnT detectable by the conventional standard cTnT assay and the novel long cTnT assay according to one embodiment of the invention. The black tracer antibodies are marked with stars, while capture antibodies are white in the figure.

FIG. 2 shows the long/standard cTnT ratio in patients with ESRD, NSTEMI and STEMI. Long cTnT was determined with an immunoassay that uses three tracer antibodies.

FIG. 3 shows the standard cTnT, long cTnT and long/standard cTnT ratio in patients with ESRD and NSTEMI patients with ≤24 h hours delay between symptom onset and sample collection. Long cTnT was determined with an immunoassay that uses three tracer antibodies.

FIG. 4 shows the relation of long/standard cTnT ratio and time from symptom onset to blood sampling in patients with NSTEMI and STEMI. The decrease in the ratio followed typical exponential decay function with an elimination half-time of 13.9 h (95% confidence intervals shown as dotted lines, elimination constant 0.0499, standard error 0.0075, r=0.613). Long cTnT was determined with an immunoassay that uses three tracer antibodies.

FIG. 5 shows long (on the left) and standard (on the right) cTnT before and immediately after hemodialysis in the ESRD group. Long cTnT was determined with an immunoassay that uses three tracer antibodies.

FIG. 6 shows receiver operating characteristic curves depicting the discriminative capacity of cTnT ratio (long/standard) (full line) and standard cTnT (dotted line) between ESRD and NSTEMI (all patients: CTnT ratio AUC 0.906 (CI95% 0.837-0.975) and standard cTnT AUC 0.688 (CI95% 0.575-0.800) [panel A]; NSTEMI patients with a ≤24 h delay between symptom onset and sample collection: cTnT ratio AUC 0.955 (CI95% 0.899-1.0) and standard cTnT AUC 0.609 (CI95% 0.454-0.764) [panel B]). Long cTnT was determined with an immunoassay that uses three tracer antibodies.

FIG. 7 shows the standard cTnT, long cTnT and long/standard cTnT ratio in patients with ESRD and NSTEMI patients with ≤24 h hours delay between symptom onset and sample collection when long cTnT is determined with an immunoassay that uses only one tracer antibody.

### DETAILED DESCRIPTION

Before the invention is described, it is to be understood that this disclosure is not strictly limited to any particular compositions, reagents, antibodies, devices, protocols or methodology described herein, as such may vary. It is also to be understood that the terminology used herein is for the purpose of describing particular embodiments only, and is not intended to be limiting, since the scope of the present invention will be limited only by the appended claims.

It is also to be noted that, unless defined otherwise, all technical and scientific terms used herein have the same meaning as commonly understood by one of ordinary skill in the art to which this invention belongs.

It is further to be noted that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable subcombination. Moreover, any features, details or embodiments disclosed in the context of a method provided herein apply also to a kit provided herein, and vice versa, even if not repeated. Correspondingly, any such features, details or embodiments apply also to various uses of the kit even if not repeated.

As used herein and in the appended claims, the singular forms “a”, “an” and “the” mean one or more. Thus, a singular noun, unless otherwise specified, carries also the meaning of the corresponding plural noun, and vice versa. As such, the terms “a”, “an”, “one or more” and “at least one” can be used interchangeably.

The term “and/or” in a phase such as “X and/or Y” shall be understood to mean either “X and Y” or “X or Y” and shall be taken to provide explicit support for both meanings or for either meaning.

The terms “comprising”, “including” and “having” can be used interchangeably, and they are intended to be construed in a non-exclusive manner, i.e., allowing for items, components or elements not explicitly described also to be present.

As used herein, the term “myocardial infarction” (MI) refers to an extremely dangerous condition, commonly called a heart attack, caused by a lack of sufficient blood flow to the heart muscle. Acute MI results from acute obstruction of a coronary artery. Common symptoms of MI include, for example, chest pain or discomfort, abnormal heart beating, nausea, vomiting, shortness of breath and fatigue. Diagnosis of MI is by electrocardiogram (ECG) and the presence or absence of serologic markers, especially cardiac troponins.

As used herein, the term “ST elevation myocardial infarction” (STEMI) refers to a type MI with a pattern known as ST elevation on the ECG. Measurement of cardiac troponins is not necessary for diagnosis, although cardiac troponins are usually analysed when the situation allows.

As used herein, the term “non-ST elevation myocardial infarction” (NSTEMI) refers to a type of MI diagnosed on the basis of symptoms consistent with MI and elevated cardiac troponins without ECG changes indicative of a STEMI.

As used herein, the term “end-stage renal disease” (ESRD), also known as end-stage renal failure, is the final, permanent stage of chronic kidney disease (CKD). Symptoms of CKD and ESRD include, for example, chest pain, nausea, vomiting, shortness of breath, fatigue and weakness. Elevated high-sensitivity cardiac troponin T levels are common in CKD and ESRD patients. Thus, diagnosing NSTEMI in CKD and ESRD patients is challenging.

As used herein, the term “cardiac troponin T” (cTAT) refers to one of the subunits of human cardiac troponin, a myofibrillar protein that is present in the heart tissue, and plays an important role in regulating muscle contraction by regulating the binding of tropomyosin and actin. Cardiac troponin T is considered a powerful marker of acute myocardial infarction. However, elevated cTnT levels are present in patients with impaired renal function and can be seen in other conditions as well.

Human cardiac troponin T is encoded by the TNNT2 gene. At least ten different isoforms of cTnT have been identified, which result from alternative splicing of the mRNA transcript. The dominant isoform found in normal adult human heart tissue is isoform 6 also known as TNT3 (UniProtKB/Swiss-Prot entry P45379-6). Isoform 6 has a calculated molecular weight of about 34.6 kDa, and its amino acid sequence is as set forth in SEQ ID NO: 1 consisting of 288 amino acids.

It is to be understood that although isoform 6 is used herein as a reference cTnT, the invention is not limited thereto. Thus, any and all references to certain cTnT fragments, for example, do not mean those of isoform 6 only but encompass also corresponding fragments of other cTnT isoforms existing in human heart after birth. Likewise, the amino acid sequence set forth in SEQ ID NO: 1 represents a reference cTnT sequence derived from isoform 6. Thus, any reference to a particular subsequence or amino acid residue of SEQ ID NO: 1 encompasses corresponding subsequences or amino acid residues in other relevant cTnT isoforms.

Cardiac TnT is cleaved in vivo resulting in cTnT fragments of different sizes. The predominant cTnT cleavage sites have been documented to locate between amino acid residues corresponding to amino acids 68 and 69 in SEQ ID NO: 1, in the C-terminal cleavage area around amino acid residues corresponding approximately to amino acids 189-223 in SEQ ID NO: 1 with multiple cleavage sites, and between amino acid residues 287 and 288 in SEQ ID NO: 1. N-terminal cleavage results in N-terminal short fragment (˜8 kDa, aa 1-68 in SEQ ID NO:1) and C-terminal long fragment (˜29 kDa, aa 69-287 or aa 69-288 in SEQ ID NO:1). C-terminal cleavage results in N-terminal long fragment (˜22-30 kDa) and C-terminal short fragment (˜8-15 kDa). Owing to the presence of multiple potential cleavage sites in the C-terminal cleavage area exact amino acid boundaries of the N-terminal long fragment and the C-terminal short fragment may vary. Cleavage at both predominant cleavage sites results in N-terminally and C-terminally truncated mid-fragment (˜14-20 kDa, the predominant 16 kDa fragment representing aa 69-189 in SEQ ID NO:1). The indicated molecular weights refer those of cTnT isoform 6 approximated on the basis of Western blotting. Also the indicated amino acid boundaries refer to the amino acid boundaries of cTnT isoform 6. It is thus to be understood that the molecular weights and amino acid boundaries may vary depending on the cTnT isoform in question.

As used herein, the term “long cTnT” refers to cTnT forms that are not cleaved at the predominant C-terminal cleavage area corresponding to amino acids 189-223 in SEQ ID NO: 1, and contain a C-terminal region corresponding to amino acids 205-215 in SEQ ID NO: 1. These forms may herein be referred to as a first set of cTnT molecules. The term thus includes both intact or full-length cTnT (˜37 kDa) and the C-terminal long fragment (˜29 kDa), as well as possible other fragments comprising the regions set forth above. However, the first set of cTnT molecules does not include any C-terminally truncated fragments, i.e. fragments cleaved at the C-terminal region corresponding to amino acids 189-223 of SEQ ID NO: 1, including C-terminal short fragments (˜8-15 kDa), N-terminal short fragments (˜8 kDa) and both N-terminally and C-terminally truncated mid-fragment (˜14-20 kDa).

As used herein, the term “intact cTnT” refers to a substantially full-length cTAT molecule (˜37 kDa) that is either non-cleaved or cleaved only at the C-terminal cleavage site positioned between the last two amino acids corresponding to amino acids 287 and 288 in SEQ ID NO: 1. In other words, “intact cTnT” has an amino acid sequence corresponding to amino acids 1-287 or 1-288 in SEQ ID NO: 1.

As used herein, the term “standard cTnT” refers to cTnT forms that are detectable by the gold standard high-sensitivity cTnT (hs-cTnT) assay commercially available by Roche Diagnostics, or by any corresponding cTnT assay. The Roche assay works according to the sandwich principle, in which a detector (M7) and catcher (M11.7) antibody are allowed to bind to cTnT, forming a sandwich complex. Since both antibodies bind closely to one another at the central part of the cTnT molecule, the assay is able to detect all cTnT isoforms and fragments thereof containing the epitope sequences corresponding to these antibodies. Accordingly, the term “standard cTnT” includes intact cTnT (˜37 kDa), C-terminal long fragment (˜29 kDa), N-terminal long fragment (˜22-30 kDa) as well as N-terminally and C-terminally truncated mid-fragment (˜14-20 kDa). In other words, the term includes all other cTnT fragments mentioned above except the N-terminal short fragment (˜8 kDa) and the C-terminal short fragment (˜8-15 kDa). For the sake of simple expression, cTnT forms denoted as “standard cTnT” may herein be also be referred to collectively as a second set of cTnT molecules.

As used herein, the term “long/standard cTnT ratio” refers to calculated ratio of determined concentration of long cTnT and determined concentration of standard cTnT, i.e. long cTnT concentration divided by standard cTnT, and optionally multiplied by 100% to express the ratio as a percentage value. In other words, the “long/standard cTnT ratio” defines which proportion of standard cTnT consists of long cTnT in a given sample. For example, ratios 0.5 and 50% both mean that half of the standard cTnT consists of long cTnT. However, depending on the calibration of the assays and possible assay specific interferences, in some instances the ratio may be above 1 or 100%.

As used herein, the term “standard/long cTnT ratio” refers to a calculated ratio of determined concentration of standard cTnT and determined concentration of long cTnT, i.e. standard cTnT concentration divided by long cTnT, and optionally multiplied by 100% to express the ratio as a percentage value. In other words, the “standard/long cTnT ratio” defines the relative proportion of standard cTnT to long cTnT in a given sample. For example, ratios 2 and 200% both mean that the concentrations of standard cTnT is double the concentration of long cTnT and that the half of the standard cTnT consists of long cTnT.

As used herein, the term “third set of cTnT molecules” refers collectively to “C-terminal cTAT molecules”, i.e. all cTnT forms that contain the C-terminal part of cTnT, namely the region ranging from the C-terminal cleavage area (aa 189-223 of SEQ ID NO: 1)) to the C-terminal end (aa 287 or 288 of SEQ ID NO: 1). Such molecules include intact cTnT (˜37 kDa), C-terminal long fragment (˜29 kDa) and C-terminal short fragment (˜8-15 kDa), but not N-terminal long fragment (˜22-30 kDa), N-terminal short fragments (˜8 kDa), or N-terminally and C-terminally truncated mid-fragment (˜14-20 kDa).

As used herein, the term “diagnosing” refers broadly, without limitation, to a process aimed at determining or assessing the probability whether or not a subject is afflicted with a disease or condition such as acute myocardial infarction. This is also meant to include instances where the presence of the disease or condition is not finally determined but that further diagnostic testing is warranted. In such embodiments, the method is not by itself determinative of the presence or absence of said disease or condition but can indicate that further diagnostic testing is needed or would be beneficial. Diagnostic test results may also exclude the presence of a given disease or condition without final diagnosis. In some instances, diagnostic test results may also be used to strengthen a diagnosis already performed using other indicators. Therefore, the present method may be combined with one or more other diagnostic methods for the final determination of the presence or absence of said disease or condition in the subject. Such other diagnostic methods are well known to a person skilled in the art, including but not limited to, ECG.

As used herein, the term “subject” refers to an animal subject, preferably to a mammalian subject, more preferably to a human subject. Herein, the terms “subject”, “individual” and “patient” are interchangeable, especially when concerning human subjects.

As used herein, the term “sample” refers in particular to a blood sample, such as a serum or plasma sample, obtained from a subject. However, plasma samples, such as heparin plasma samples, EDTA plasma samples and the like, are preferred because thrombin that is present in normal serum is known to cleave cTnT at the N-terminal cleavage site between amino acid residues corresponding to amino acids 68 and 69 in SEQ ID NO: 1. Generally, obtaining the sample from the subject is not part of the assays or methods disclosed herein, rendering them as an in vitro assays or methods to be carried out with samples taken from the patient earlier.

As used herein, “sensitivity” is a measure of the ability of a marker, such as cTnT, to detect a particular disease or condition, such as MI. In other words, sensitivity represents the probability of a positive test result in subjects with the disease or condition.

As used herein, the term “true positive” (TP) refers to a test result which classifies a subject who has a particular disease or condition correctly as a subject having the disease or condition. Likewise, “true negative” (TN) refers to a test result which classifies an unaffected subject correctly as an unaffected.

As used herein, “specificity” is a measure of the ability of a marker to detect the absence of a particular disease or condition. In other words, specificity represents the probability of a negative test result in a subject without the disease or condition.

As used herein, the term “false positive” (FP) refers to a test result which classifies an unaffected subject incorrectly as an affected subject, i.e., as a subject with the disease or condition. Likewise, “false negative” (FN) refers to a test results which classifies a subject with a particular disease or condition incorrectly as an unaffected subject.

As used herein, the term “accuracy”, also called “diagnostic effectiveness”, refers to a proportion of correctly classified subjects (TP+TN) among all subjects (TP+TN+FP+FN).

Receiver Operating Characteristic (ROC) curves may be utilized to demonstrate the trade-off between the sensitivity and specificity of a marker, as is well known to those skilled in the art. The horizontal X-axis of the ROC curve represents 1—specificity, which increases with the rate of false positives. The vertical Y-axis of the curve represents sensitivity, which increases with the rate of true positives. Thus, for a particular cut-off (i.e., threshold) selected, the values of specificity and sensitivity may be determined. In other words, data points on the ROC curves represent the proportion of true-positive and false-positive classifications at various decision boundaries. Optimum results are obtained as the true-positive proportion approaches 1.0 and the false-positive proportion approaches 0.0. However, when the cut-off is changed to increase specificity, sensitivity usually is reduced, and vice versa.

The area under the ROC curve, often referred to as the AUC, is a measure of the utility of a marker in the correct identification of disease subjects, i.e., subjects who are affected by a disease or condition. Thus, the AUC values can be used to determine the effectiveness of the test. An area of 1.0 represents a perfect test; an area of 0.5 represents a worthless test. A traditional rough guide for classifying the accuracy of a diagnostic test is the following: AUC values 0.9 to 1.0 represent a test with excellent diagnostic power, AUC values 0.80 to 0.90 represent a test with good diagnostic power, AUC values 0.70 to 0.80 represent a test with fair diagnostic power, AUC values 0.60 to 0.70 represent a test with poor diagnostic power, and AUC values 0.50 to 0.60 represent a test with failed diagnostic power.

As used herein, the term “binder molecule” refers broadly to any agent that specifically binds to its target epitope. The term includes, without limitation, antibodies as well as non-antibody scaffold formats such as, but not limited to, affibodies and aptamers.

As used herein, the term “antibody” refers generally to an immunoglobulin structure comprising two heavy (H) chains and two light (L) chains interconnected by disulphide bonds, including but not limited to polyclonal, monoclonal, and recombinant antibodies of isotype classes IgA, IgD, IgE, IgG, and IgM and subtypes thereof. Means and methods for producing antibodies are readily available in the art.

Antibodies can exist as intact immunoglobulins or as any of a number of well-characterized antigen-binding fragments or single chain variants thereof, all of which are herein encompassed by the term “antibody”. Non-limiting examples of said antigen-binding fragments include Fab fragments, Fab′ fragments, F(ab′)2 fragments, Fv fragments, scFv fragments (i.e., single-chain variable fragments), and nanobodies (i.e., monomeric variable domains of camelid heavy chain antibodies), as well as such fragments when engineered to form fusions with FC region. Antigen-binding fragments and single-chain variants may be produced by recombinant DNA techniques, or by enzymatic or chemical separation of immunoglobulins as is well known in the art.

As used herein, the term “epitope” refers broadly to a part of a target protein that is specifically recognized by an antibody or other binder molecule. As is well known in the art, some epitopes may be discontinuous, i.e., composed of several small fragments that are scattered in the amino acid sequence possibly even in different subunits or polypeptide chains of the protein, but are close in the three-dimensional structure of the protein. Sometimes an epitope can be formed of amino acids from different proteins when they become in close proximity, for example owing to complex formation. Such epitope cannot be defined through linear amino acid sequence.

As used herein, the term “specific binding”, “specifically binding” or “is specific” refers to the discriminatory binding of a binder molecule to its target sequence or epitope such that binder molecule does not substantially cross-react with non-target sequences, i.e., does not have significant non-specific binding.

As readily understood by those skilled in the art, binder molecules do not have to be 100% complementary to their target sequences provided that they still specifically bind to the target epitopes. Accordingly, the binder molecules may still bind to their target epitopes even if some amino acids either at the N-terminal or C-terminal end of the epitope region are missing, for example owing to the cleavage of the cTnT molecule.

As used herein, the term “quantitating”, and any corresponding expressions, refer to quantifying or measuring the amount of cTnT or a set of cTnT molecules, such as long cTnT or standard cTnT, in a sample. The term “amount” is interchangeable with the terms “level” and “concentration”, and can refer to an absolute or relative quantity.

### DETAILED DESCRIPTION

The present invention relates broadly to a new generation troponin assay, more specifically to an assay for detecting long cTnT in a sample, and in particular to an assay for determining the ratio of long cTnT and standard cTnT in a sample. The assays have improved specificity for acute MI over conventional high-sensitivity standard cardiac troponin T (hs-cTnT) assays, owing to standard cardiac troponin T being commonly elevated also in conditions other than MI, including for example acute or chronic renal dysfunction, atrial fibrillation and strenuous exercise.

The assay comprises quantitating the amount of long cTnT in a sample by contacting the sample with at least one first binder molecule capable of specific binding to the C-terminal part of cTnT, more specifically to a region corresponding to amino acids 201-288, preferably amino acids 223-288 of SEQ ID NO:1 and with at least one second binder molecule capable of specific binding to a part towards the N-terminus, more specifically to a region corresponding to amino acids 1-200, preferably amino acids 1-190, 69-200, 69-190, 164-200 or 164-190 of SEQ ID NO:1. Surprisingly, the first binder molecule can also be selected among binder molecules that are capable of binding specifically to cardiac troponin I (cTnI; SEQ ID NO: 2), cardiac troponin C (cTnC; SEQ ID NO: 3) or to an epitope that is formed when the long cTnT is in complex with cTnI and cTnC or when cTnI and cTnC are in complex with each other. Binding of both the at least one first and the at least one second binder molecule to the same troponin T molecule discriminates long cTnT molecules from other common cTnT fragments possibly present in the sample, including the most prominent N-terminal long fragments, N-terminally and C-terminally truncated mid-fragments and C-terminal short fragments. Concentration of long cTnT in the sample correlates with the level of a binding reaction involving both the first and the second binder molecule. In some embodiments, number of the first and the second binder molecules varies, independently from each other, usually from 1 to 3, i.e., is typically 1, 2 or 3.

In some other embodiments, the assay comprises quantitating the amount of long cTnT in a sample by contacting the sample with at least one first binder molecule capable of specific binding to intact C-terminal cleavage area of cTnT, more specifically to a region corresponding to amino acids 195-220 of SEQ ID NO: 1, and whose binding to its epitope is severely impaired by cleavage at the cleavage area; and with at least one second binder molecule capable of specific binding either to a part towards the N-terminus, more specifically to a region corresponding to amino acids 1-194, preferably amino acids 1-190, 69-194, 69-190, 164-194 or 164-190 of SEQ ID NO:1, to a part towards the C-terminus, more specifically to a region corresponding to amino acids 221-288, preferably to amino acids 221-287, 224-287 or 224-288 of SEQ ID NO: 1, to cTnI (SEQ ID NO: 2), to cTnC (SEQ ID NO: 3), or to an epitope that is formed when the long cTnT is in complex with cTnI and cTnC or when cTnI and cTnC are in complex. Since, by definition, the C-terminal cleavage area of long cTnT is intact, such first binder molecule can positively discriminate long cTnT from other forms of cTnT molecules (i.e., cTnT fragments which have been cleaved at the C-terminal cleavage area) that may exist in a sample whose long cTnT content is to be quantitated. Thus, also in these embodiments, binding of both the at least one first and the at least one second binder molecule to the same troponin T molecule (or cTnI or cTnC in complex with that troponin T molecule) discriminates long cTnT molecules from other common cTnT fragments possibly present in the sample, including the most prominent N-terminal long fragments, N-terminally and C-terminally truncated mid-fragments and C-terminal short fragments. Concentration of long cTnT in the sample correlates with the level of a binding reaction involving both the first and the second binder molecule. In some embodiments, number of the first and the second binder molecules varies, independently from each other, usually from 1 to 3, i.e., is typically 1, 2 or 3.

In some embodiments, the first binder molecule is a capture molecule, such as a capture antibody, and the second binder molecule is a tracer molecule, such as a tracer antibody, or vice versa. In some embodiments, more than one (usually 1, 2 or 3) capture and/or tracer molecules may be employed to improve detection. In some embodiments, one capture molecule is used in combination with one, two or three tracer molecules.

In some embodiments, the first binder molecule is specific to a cTnT epitope corresponding to amino acids 223-242 of SEQ ID NO: 1. In some further embodiments, the second binder molecule is specific to a cTnT epitope selected from the group consisting of epitopes corresponding to amino acids 67-86, amino acids 119-138, amino acids 125-130, amino acids 132-151, amino acids 136-147, amino acids 145-164, amino acids 171-190 of SEQ ID NO: 1. In some other embodiments, the first binder molecule can be an anti-cTnC antibody targeting amino acids 1-100 or full length protein, or an anti-cTnI antibody specific to a cTnI epitope selected from a group consisting of epitopes corresponding amino acids 2-16, 14-23, 19-23, 19-29, 19-36, 23-32, 23-41, 24-30, 25-41, 41-49, 42-50, 87-91, 26-41, 27-36, 35-38, 42-50, 66-75, 84-94, 87-91, 88-91, 118-127, 131-146, 137-148, 170-179, 187-193, 191-197, 196-210 of SEQ ID NO: 2. Both anti-cTnC antibodies and cTnT antibodies are commercially available from several vendors, including for example HyTest, Novus Biologicals, and Thermo Fisher Scientific.

Also antibodies specific for epitopes that are formed when the long cTnT is in complex with cTnI and cTnC, or when cTnI and cTnC are in complex with each other are commercially available, for example from HyTest.

In an assay according to some embodiments, the first binder molecule is a capture molecule, preferably a capture antibody, which is specific to a cTnT epitope corresponding to amino acids 223-242 of SEQ ID NO: 1, and the second binder molecule is a tracer molecule, preferably a tracer antibody, which is specific to a cTnT epitope selected from epitopes corresponding to amino acids 67-86, 119-138 or 171-190 of SEQ ID NO: 1. In some embodiments, the assay may employ two such tracer molecules, or all three.

Accordingly, the amount of long cTnT in a sample can be determined based on the extent of a binding reaction involving both a first and a second binder molecule with the help of previous measurements of standard solutions of known troponin concentrations. In other words, quantitating long cTnT may involve use of a calibration curve, i.e., a standard curve. Generating calibration curves is well known in the art, and can be carried out by plotting the rate of the binding reaction versus the concentration of troponin that was applied in a given standard solution. Any unknown concentration of the long cTnT may then be determined by comparing the detected rate of the binding reaction to a corresponding rate on the calibration curve, achieved in the presence of known troponin concentrations.

Thus, after quantitating the amount of long cTnT in a test sample, the measured amount of said long cTnT can be compared with any appropriate reference amount of long cTnT, such as the concentration of long cTnT derived from one or more apparently healthy subjects. In some embodiments, the reference amount is relative to an amount derived from population studies, preferably carried out in normal apparently healthy population. A reference value derived from one or more apparently healthy subjects or from an apparently healthy population may be denoted as “a normal reference amount”. General considerations, such as age range, gender, ethnicity and the like may be taken into consideration when selecting an appropriate reference amount. Result of the comparison may then be used in clinical decision making, for example to identify a patient suspected of having acute MI either as a patient probably actually having acute MI or as probably not having acute MI, and to choose appropriate treatment procedures.

Surprisingly, diagnostic performance of the present assay is even more prominent when the assay includes calculating either the ratio of long cTnT to the standard cTnT measurable by conventional high-sensitivity cTnT (hs-cTnT) assays (long/standard cTnT ratio) or the ratio of the standard cTAT to the long cTnT (standard/long cTnT ratio) (discrimination between NSTEMI with sample taken within 24 h after the symptom vs. ESRD patients, AUC for long cTnT 0.937 (CI95% 0.874-1.0) and for long cTnT to standard cTnT ratio 0.955 (CI95% 0.899-1.0)). The conventional gold standard hs-cTnT assay commercially available by Roche Diagnostics uses a capture antibody that recognizes the epitope at amino acids 136-147 of SEQ ID NO: 1 and the tracer antibody conjugated with a detectable label that recognizes the epitope at amino acids 125-130 of SEQ ID NO: 1. Thus, the hs-cTnT assay targets the middle (i.e., central) part of the cTnT molecule detecting all longer and shorter cTnT molecules that contain this part.

Notably, the assay of the invention may in some embodiments utilize the hs-InT assay by Roche Diagnostics, or any other already available equivalent hs-InT assay, to quantitate standard cTnT in a sample to be analysed. However, the assay of the invention is not limited to the utilization of any available hs-InT assay, such as the one by Roche Diagnostics. Instead, in some embodiments, any one or more binder molecules capable of specifically targeting and detecting the central part of the human cTnT may be employed in the present assay to quantitate standard cTnT.

Accordingly, quantitating standard cTnT in a sample to be analysed may be carried out by contacting the sample with a third and a fourth binder molecule both specifically binding to a region corresponding to amino acids 69-189 of SEQ ID NO: 1, preferably to a region corresponding to amino acids 69-160 of SEQ ID NO: 1. Preferably, the third binder molecule is a capture molecule, such as a capture antibody, and the fourth binder molecule is a tracer molecule, such as a tracer antibody, or vice versa. In some embodiments more than one capturer and/or tracer molecule may be employed to improve detection. Usually the number of capture and/or tracer molecules varies from 1 to 3, i.e., is typically 1, 2 or 3.

In some embodiments, the third and the fourth binder molecules are specific to different cTnT epitopes selected from the group consisting of epitopes corresponding to amino acids 67-86, amino acids 119-138, amino acids 125-130, amino acids 132-151, amino acids 136-147, amino acids 145-164, amino acids 171-190 of SEQ ID NO: 1.

As an alternative, or in addition, to quantitating standard cTnT, the assay method may in some embodiments involve quantitating a third set of cTnT molecules that includes all cTnT forms that contain the C-terminal part of cTnT, namely the region ranging from the C-terminal cleavage area to the C-terminal end. Such forms may herein be collectively denoted as “C-terminal cTnT”. In some embodiments, the third set of cTnT molecules is to be quantitated with the aid of a fifth and a sixth binder molecule specifically binding to different epitopes in a region corresponding to amino acids 223-288 of SEQ ID NO: 1. Alternatively, the sixth binder molecule can be selected among binder molecules that are capable of binding specifically to cardiac troponin I (cTnI; SEQ ID NO: 2), cardiac troponin C (cTnC; SEQ ID NO: 3) or to an epitope that is formed when the cTnT form to be quantitated is in complex with cTnI and cTnC or when cTnI and cTnC are in complex with each other. Some non-limiting examples of suitable cTnI epitopes are listed above. Preferably, the fifth binder molecule is a capture molecule, such as a capture antibody, and the sixth binder molecule is a tracer molecule, such as a tracer antibody, or vice versa. In some embodiments more than one capture and/or tracer molecule may be employed to improve detection. Usually the number of capture and/or tracer molecules varies from 1 to 3, i.e., is typically 1, 2 or 3.

Just like in the case of quantitating long cTnT (i.e., a first set of cTnT molecules), quantitating standard cTnT (i.e., a second set of cTnT molecules) or C-terminal cTnT (i.e., a third set of cTnT molecules) may involve utilizing appropriate standard curves to determine the relative or absolute amount of standard cTnT or C-terminal cTnT in a sample.

The ratio of long cTnT to standard cTnT, or the ratio of long cTnT to C-terminal cTnT, may be calculated by simply dividing the measured amount of long cTnT by the measured amount of standard cTnT or C-terminal cTnT, respectively. It also possible to use reversed ratios calculated by dividing the measured amount of standard cTnT or the measured amount C-terminal cTnT by the measured amount of long cTnT. The calculated ratio may then be compared with any corresponding appropriate reference ratio, and used in clinical decision making, for example to identify a patient whose sample has been analysed with the present assay either as a patient probably having acute MI or as probably not having acute MI.

Just like in the case of appropriate reference amounts, the corresponding appropriate reference ratio (long/standard cTnT ratio, long/C-terminal cTnT ratio, standard/long cTnT ratio, or C-terminal/long cTnT ratio as the case may be) may be obtained from one or more apparently healthy subjects. In some embodiments, the reference ratio is relative to a ratio derived from population studies, preferably carried out among normal apparently healthy population. A reference ratio derived from apparently healthy subjects or an apparently healthy populations may be denoted as “a normal reference ratio”. General considerations, such as age range, gender, ethnicity and the like may be taken into consideration when selecting an appropriate reference ratio.

It is to be understood that any appropriate reference amount or reference ratio, collectively denoted as a reference value, may refer either to an absolute value or to a relative. Moreover, any reference value may be a range value. Therefore, a reference values to be employed may be, for example, an absolute value or an absolute range value, or a relative value or a relative range value.

Generally, an increase in the measured long cTAT concentration or in the long/standard cTAT ratio as compared to a corresponding normal reference value or a corresponding normal reference range is indicative of the presence of M1. However, when a standard/long cTnT ratio is to be used, a decrease in the measured ratio as compared to a corresponding normal reference ratio or a corresponding normal reference ratio range is generally indicative of the presence of MI. In some embodiments, a deviation (either increase or decrease as explained above) of at least 5%, at least 10%, at least 20%, at least 30%, at least 40%, at least 50%, at least 60%, at least 70%, at least 80%, at least 90%, at least 100% or more in the measured long cTnT concentration, long/standard cTAT ratio or standard/long cTnT ratio as compared to a corresponding normal reference value or range may be considered as indicative of the present of MI. Accordingly, no significant deviation (neither increase nor decrease as explained above) in the measured long cTnT concentration, long/standard cTnT ratio or standard/long cTAT ratio as compared to a corresponding normal reference value or range may be considered as indicative of the absence of MI, despite elevated standard cTnT.

As an alternative of or in addition to a normal reference value and/or a normal reference range, the reference value or the reference range value may in some embodiments have been derived from one or more subjects having a known disease status, such as NSTEMI or STEMI with a known delay time between symptom onset and blood sample collection, or a known renal dysfunction status.

In some embodiments, the reference value may be a value obtained from the same subject obtained at an earlier point of time. Such embodiments allow monitoring for any change either in the subject's long cTnT concentration, long/standard cTnT ratio or standard/long cTAT ratio over time. Generally, no significant change over time is indicative of elevated standard cTnT levels being caused by a reason other than MI.

It is to be understood that the calculated cTnT concentrations and ratios as well the reference concentrations and ratios may vary depending on various variables, such as the assay format, the type and number of binder molecules used, the calibration of the assay, and the detection technique used for measuring the concentration of the cTnT form(s) in questions.

The present assay may be of any available noncompetitive assay type, as is readily understood by those skilled in the art. Preferably the assay is a noncompetitive immunoassay. Non-limiting examples of suitable immunoassays include enzyme linked immunosorbent assays (ELISA), enzyme immunoassays (EIA), radio-immunoassays (RIA), counting Immunoassays (CIA), fluorescence immunoassays (FIA), chemiluminescent immunoassays (CLIA), electrochemiluminescent immunoassays (ECLIA), immuno-PCR assays, open sandwich immunoassays (OS) and microsphere-based immunoassays (MIA).

In some embodiments, the assay is a solid-phase immunoassay, such as a lateral flow assay or a conventional sandwich assay carried out on a solid surface, e.g., on a microtiter plate or beads. In these assay formats, a capture molecule (i.e., a first or a third binder molecule) is immobilized on the solid surface, and the tracer molecule is detectably labelled. If a test sample contains the troponin species to be quantitated, a detectable sandwich forms between the troponin analyte and the capture and tracer molecules.

Non-limiting examples of suitable solid surfaces for solid-phase assays include, but are not limited to, those based on polystyrene, nitrocellulose or nylon membranes, glass or silicon. Capture molecules can be immobilized on the solid surface using any appropriate technique available in the art including, but not limited to, surface modification techniques, covalent coupling techniques and affinity immobilization techniques. For example, biotin has strong affinity to streptavidin. Therefore, when coated on a solid surface, streptavidin can serve as an immobilizing agent for biotinylated capture molecules, such as antibodies. Further affinity immobilization techniques include, for example, DNA directed immobilization, Protein A and Protein G mediated immobilization, immobilization via Fc-binding peptides and aptamers, and immobilization through material binding peptides or through metal affinity. Covalent coupling may be achieved, for example, by targeting amine, carboxyl, thiol or carbohydrate groups in the capture molecule, or by using “click” chemistries.

The choice of the detectable label to be conjugated or otherwise associated with the tracer molecule depends on the detection technique to be used, as is readily understood by those skilled in the art. Examples suitable of detectable labels include, but are not limited to, optical agents such as fluorescent labels including a variety of organic and/or inorganic small molecules, lanthanide chelates, upconverting nanoparticles and a variety of fluorescent proteins and derivatives thereof, phosphorescent labels, chemiluminescent labels, electrochemiluminescent labels, and chromogenic labels; radioactive labels such as radionuclides that emit gamma rays, positrons, beta or alpha particles, or X-rays; and enzymes such as alkaline phosphatase (AP), or (horseradish) hydrogen peroxidase (HRP); and oligonucleotides that form an amplicon to be detected by PCR reaction. Said association can be direct, e.g., through a covalent bond, or indirect, e.g., via a secondary binding agent, chelator, or linker. Techniques for conjugating or otherwise associating detectable agents to tracer molecules, such as antibodies, are well known and for example antibody labelling kits are commercially available from dozens of sources. A tracer molecule may also be expressed as a fusion protein with a detectable label or a detection tag by recombinant techniques.

In some embodiments, fluorescent labels, especially Europium (III) chelates, are preferred. A nonlimiting example of such chelates include [2,2′,2″,2′″-{[2-(4-isothiocyanatophenyl)ethylimino]bis-(methylene)bis{4-{[4-(alfa-galactopyranoxy)phenyl]ethynyl}pyridine-6,2-diyl}bis(methylenenitrilo)}tetrakis(acetato)] europium(III) chelate.

Solid-phase immunoassays may be either heterogeneous or homogeneous. In heterogeneous assays, any free analytes or tracer molecules must be physically separated from the assay reaction, e.g., by washings, while no such separation is necessary in homogeneous assays making homogeneous assays convenient.

In some preferred embodiments, the assay of the invention is a heterogenous solid-phase immunoassay.

Homogeneous immunoassays may be carried out not only in solid-phase assay formats but also in solution. Such in-solution immunoassays are particularly advantageous because no immobilization or washing steps are required, making them simple and easy to perform. Examples of homogeneous assays include, but are not limited to, colorimetric assays, assays based on detection of ultraviolet signals, fluorescence resonance energy transfer (FRET) assays, proximity ligation immunoassays (PLA), proximity extension immunoassays (PEA), luminescent assays based on oxygen channeling (AlphaLISA and AlphaScreen). Nano-materials such as upconverting nanoparticles, noble metal nanoparticles, quantum dots (QDs), and graphene oxide (GO) may also be employed in homogeneous assays as is well known in the art.

Some embodiments may employ proximity detection (PLA) technology to detect or quantitate long cTnT and/or standard cTnT in a sample. Generally, this may be achieved by employing different DNA oligonucleotides coupled to protein recognition molecules to form PLA probes. After contacting the sample with the PLA probes, two PLA probes containing different DNA sequences will be simultaneously bound to the same cTnT molecule to be detected or quantitated. After the binding reactions, the DNA tails of the two probes are close in space, and hybridize next to each other to a third free oligonucleotide, that is complementary to the DNA sequences of the PLA probes. The 5′ and 3′ ends of the DNA tails of the two probes are ligated by ligase enzyme to form an amplicon to be quantitated in quantitative PCR reaction. The amount of ligated single-stranded DNA positively correlates with the amount of cTnT molecules to be measured in the sample.

Some embodiments may employ a competitive assay format, preferably a competitive immunoassay format, for quantitating long cTnT. In such embodiments, only one binder molecule, preferably an antibody, is used that binds specifically to the C-terminal cleavage area of cTnT and whose binding to its epitope is severely impaired by cleavage at a the cleavage area. Since, by definition, the C-terminal cleavage area of long cTnT is intact, such a binder molecule can positively discriminate long cTnT from other forms of cTnT molecules (i.e., cTnT fragments which have been cleaved at the C-terminal cleavage area) that may exist in a sample whose long cTnT content is to be quantitated. In some embodiments, for example, long cTnT competes against a detectably labelled analyte analog (i.e., a detectably labelled cTnT molecule or respective peptide comprising the whole or part of the intact C-terminal cleavage area) in binding to the binder molecule concentration dependently. In other words, the higher the long cTnT concentration in the sample, the lesser binding of the detectably labelled analyte analog to the binder molecule and consequently lower signal. As is well known to those skilled in the art, competitive assay formats other than the one exemplified above are also available and may be applied for quantitating long cTnT. For example, the analyte analog need not be detectably labelled, especially if analyte analog is immobilized on a solid surface and the binder molecule is detectably labelled.

The present invention also provides a kit and use thereof for quantitating desired cTnT species in a test sample. The kit comprises at least one first binder molecule capable of specific binding to a region of cTnT corresponding to amino acids 201-288, preferably amino acids 223-288 of SEQ ID NO:1, to cTnI, to cTnC, or to an epitope that is formed when the long cTnT is in complex with cTnI and cTnC or when cTnI and cTnC are in complex and at least one second binder molecule capable of specific binding to a region of cTnT corresponding to amino acids 1-200, preferably amino acids 1-190, 69-200, 69-190, 164-200 or 164-190 of SEQ ID NO: 1. Alternatively, the at least one first binder molecule is capable of specific binding to the C-terminal cleavage area of cTnT, more specifically to a region corresponding to amino acids 195-220 of SEQ ID NO:1, while the at least one second binder molecule is capable of specific binding either to a part towards the N-terminus, more specifically to a region corresponding to amino acids 1-194, preferably amino acids 1-190, 69-194, 69-190, 164-194 or 164-190 of SEQ ID NO:1, to a part towards the C-terminus, more specifically to a region corresponding to amino acids 221-288, preferably to amino acids 221-287, 224-287 or 224-288 of SEQ ID NO:1, to cTnI (SEQ ID NO: 2), to cTnC (SEQ ID NO: 3, or to an epitope that is formed when the long cTnT is in complex with cTnI and cTnC or when cTnI and cTnC are in complex. Only long cTnT can be bound by both the first and the second binder molecule. For solid-phase applications, the first binder molecule is a capture molecule, preferably a capture antibody, while the second binder molecule is a tracer molecule, such as a trace antibody, or vice versa. Nonlimiting example of suitable epitopes for the first and the second binder molecules are listed above.

In some embodiments, the kit may comprise one, two or three tracer molecules, preferably antibodies, each specifically binding to a different epitope in the region of cTnT corresponding to amino acids 1-200 of SEQ ID NO: 1. In some embodiments, said epitopes encompass amino acids 67-86, amino acids 119-138 or amino acids 171-190 of SEQ ID NO: 1. In some embodiments, the first binder molecule specifically binds to amino acids 223-242 of SEQ ID NO: 1.

In some embodiments, the kit may comprise reagents to quantify long cTnT using a competitive assay format. In such embodiments the kit comprises at least a seventh binder molecule capable of specifically binding to a region of cTnT corresponding to amino acids 189-223 of SEQ ID NO: 1, or corresponding to amino acids 190-223 or 195-220 of SEQ ID NO:1. In some embodiments, the kit further comprises an analyte analog, i.e., cTnT molecule or a peptide thereof comprising at least partly intact C-terminal cleavage area, to compete against long cTnT in binding to the seventh binder molecule, preferably a seventh antibody. In some embodiments, the seventh binder molecule is immobilized on a solid surface and the analyte analog is detectably labelled, or vice versa.

In some embodiments, the kit of the invention may further comprise binder molecules for quantitating standard cTnT. Accordingly, a third binder molecule and a fourth binder molecule capable of specific binding to a region of cTnT corresponding to amino acids 69-189 of SEQ ID NO: 1, preferably to a region corresponding to amino acids 69-160 of SEQ ID NO:1 be contained in the kit. Intact cTnT as well as all cTnT fragments containing epitopes for both the third and the fourth binder molecules can be quantitated collectively by the third and the fourth binder molecule. For solid-phase applications, one of the third and the fourth binder molecules is a capture molecule, preferably a capture antibody, while the other binder molecule is a tracer molecule, such as a trace antibody. Non-limiting examples of suitable epitopes for the third and the fourth binder molecules are listed above. In some embodiments, the epitope for the capture molecule encompasses amino acids 136-147 of SEQ ID NO: 1, while the epitope for the tracer molecule encompasses amino acids 125-130 of SEQ ID NO: 1, or vice versa. In some embodiments, the kit of the invention may further comprise binder molecules for quantitating C-terminal CTnT. Accordingly, a fifth binder molecule and a sixth binder molecule capable of specific binding to a C-terminal region of cTnT corresponding to amino acids 223-288 of SEQ ID NO: 1 may be contained in the kit. Intact cTnT as well as all cTnT fragments containing epitopes for both the fifth and the sixth binder molecules can be quantitated collectively by the fifth and the sixth binder molecule. Alternatively, the sixth binder molecule can be a binder molecule, preferably an antibody, capable of binding specifically to cTnI, cTnC or to an epitope that is formed when cTnT forms to be quantitated are in complex with cTnI and cTnC or when cTnC and cTnI are in complex. In such instances, intact cTnT as well as all cTnT fragments which i) contain an epitope for the fifth binder molecule and ii) are in complex with cTnI and cTnC can be quantitated collectively by the fifth and the sixth binder molecule. For solid-phase applications, one of the fifth and the sixth binder molecules is a capture molecule, preferably a capture antibody, while the other binder molecule is a tracer molecule, such as a tracer antibody.

Antibodies suitable for use in the present invention are available in the art. Further antibodies may be developed, for example, through traditional animal immunizations, or obtained from recombinant expression libraries by employing phage display techniques including, but not limited to, phage displays, ribosome displays, bacterial cell surface displays, yeast cell surface displays or mammalian cell surface displays.

The present assay and/or kit may be applied for different purposes, such as for determining the likelihood of acute MI in a patient suffering from symptoms of acute MI. In other words, a patient suspected of having acute MI can be identified either as a patient likely having acute MI or as a patient likely not having MI. Depending on the test result, the present assay may thus help in excluding the likelihood of the patient having MI or confirming the likelihood of the patient having MI. For example, if a sample obtained from the patient does not show elevation in the amount of long cTnT as compared to a normal reference value, or the ratio of long cTnT to standard cTnT is below a normal reference ratio or a predetermined cut-off limit, the patient suspected of having MI is not likely to actually have acute MI. The experimental part demonstrates that long cTnT and especially the long/standard cTnT ratio is markedly elevated in patients with acute MI, especially within 24 hours after MI.

It is also envisaged that the assay method and/or the kit may be employed for distinguishing patients having MI from patients showing elevated standard cTnT levels for a reason other than MI, including for example acute renal dysfunction, chronic kidney disease, ESRD, atrial fibrillation and strenuous exercise.

In some embodiments, the present assay method and/or kit can be applied to identifying a patient showing elevated standard cTnT levels and experiencing symptoms of acute MI either as a patient actually having or likely having acute MI or as a patient not having or likely not having acute MI despite the symptoms. Such an assay method provides a much-needed improved tool, especially for emergency departments, since the standard high-sensitivity cTnT assay lacks sufficient diagnostic performance in patients with transiently or chronically elevated high-sensitivity cardiac troponin.

In some embodiments, the assay method and/or the kit may be employed for distinguishing patients having MI from patients having renal dysfunction, such as CKD or ESRD, when the patients show elevated standard cTnT. Indeed, the experimental part shows that patients with chronic kidney disease, especially ESRD, show only very low concentrations of long cTnT, as well as very low long/standard cTnT ratio.

The above-described aspects and/or embodiments of the invention may be expressed in different ways. It is to be noted that all alternative and/or preferred amino acid sub-sequences referred to above may be applied for the embodiments described below even though they are not repeated.

At its simplest, provided herein is an in vitro assay method of detecting and/or quantitating long cTnT in a sample, the method comprising:
A)
i) contacting the sample with a first binder molecule specifically binding to a region of cTnT corresponding to amino acids 201-288 of SEQ ID NO:1, to cTnI, to cTnC, or to an epitope that is formed when the long cTnT is in complex with cTnI and cTnC or when cTnI and cTnC are in complex, and a second binder molecule specifically binding to a region of cTnT corresponding to amino acids 1-200 of SEQ ID NO:1; and
ii) detecting that long cTnT is present in the sample if a specific binding reaction involving both the first and the second binder molecule occurs, or quantitating long cTnT on the basis of a binding reaction in step i), preferably with the aid of a calibration curve; or
B)
i) contacting the sample with a first binder molecule specifically binding to a region of cTnT corresponding to amino acids 195-220 of SEQ ID NO:1, and a second binder molecule specifically binding to a region of cTnT corresponding to amino acids 1-194 of SEQ ID NO:1, to a region of cTnT corresponding to amino acids 221-288, to cTnI, to cTnC or to an epitope that is formed when the long cTnT is in complex with cTnI and cTnC or when cTnI and cTnC are in complex; and
ii) detecting that long cTnT is present in the sample if a specific binding reaction involving both the first and the second binder molecule occurs, or quantitating long cTnT on the basis of a binding reaction in step i), preferably with the aid of a calibration curve.

Quantification result obtained in step A or B may then be compared to an appropriate reference value, for example to determine whether the subject whose sample has been quantitated for long cTnT is likely to have or not to have MI, turning the method into an in vitro method of determining likelihood of MI in a subject. In accordance with the present invention, elevated amount of long cTnT as compared to a normal reference value is indicative of MI.

Accordingly, the above method may also be expressed as an in vitro assay method of determining likelihood of acute myocardial infarction (MI) in a subject, the method comprising:
i) measuring the amount of long cTnT in a sample obtained from the subject, the first set of cTnT molecules consisting of cTnT forms that are not cleaved at a region corresponding to amino acids 189-223 of SEQ ID NO: 1 and comprise a region corresponding to amino acids 205-215 SEQ ID NO: 1; and
ii) determining the likelihood of acute MI in the subject on the basis of the measurement.

In some embodiments, the method of determining likelihood of acute MI in a subject comprises:
i) measuring the amount of long cTnT in a sample by contacting the sample with a first binder molecule specifically binding to a region of cTnT corresponding to amino acids 201-288 of SEQ ID NO:1, to cTnI, to cTnC, or to an epitope that is formed when the long cTnT is in complex with cTnI and cTnC or when cTnI and cTnC are in complex, and with a second binder molecule specifically binding to a region of cTnT corresponding to amino acids 1-200 of SEQ ID NO:1; or
i′) measuring the amount of long cTnT in a sample by contacting the sample with a first binder molecule specifically binding to a region of cTnT corresponding to amino acids 195-220 of SEQ ID NO:1, and with and a second binder molecule specifically binding to a region of cTnT corresponding to amino acids 1-194 of SEQ ID NO: 1, to a region of cTnT corresponding to amino acids 221-288, to cTnI, to cTnC or to an epitope that is formed when the long cTnT is in complex with cTnI and cTnC or when cTnI and cTnC are in complex; and
ii) determining the likelihood of acute MI in the subject on the basis of the measurement.

In some further embodiments, the method of determining likelihood of acute MI in a subject comprises:
i) measuring the amount of long cTnT in a sample by contacting the sample with a binder molecule specifically binding to the C-terminal cleavage area and with an analyte analog, preferably cTnT or a peptide thereof comprising an intact C-terminal cleavage area or a part thereof, wherein the long cTnT present in the sample competes against the analyte analog in binding to the binder molecule; and
ii) determining the likelihood of acute MI in ii) the subject on the basis of the measurement.

In some preferred assay formats, the analyte analog is detectably labelled.

Also provided is an in vitro method to calculate the ratio of long cTnT and standard cTnT in a sample. Such a method may comprise:
i) measuring the amount of long cTnT in the sample;
ii) measuring the amount of standard cTnT in the sample; and
iii) calculating either the ratio of long cTnT to standard cTnT or the ratio of standard cTnT to long cTnT in the sample on the basis of the measurements in step i) and ii).

In some embodiments, the method of calculating the ratio of long cTnT to standard cTnT in a sample may comprise:
i) measuring the amount of long cTnT in the sample by contacting the sample with a first binder molecule specifically binding to a region of cTnT corresponding to amino acids 201-288 of SEQ ID NO:1, to cTnI, to cTnC, or to an epitope that is formed when the long cTnT is in complex with cTnI and cTnC or when cTnI and cTnC are in complex, and a second binder molecule specifically binding to a region of cTnT corresponding to amino acids 1-200 of SEQ ID NO:1; or
ii) i′) measuring the amount of long cTnT in a sample by contacting the sample with a first binder molecule specifically binding to a region of cTnT corresponding to amino acids 195-220 of SEQ ID NO:1, and with and a second binder molecule specifically binding to a region of cTnT corresponding to amino acids 1-194 of SEQ ID NO: 1, to a region of cTnT corresponding to amino acids 221-288, to cTnI, to cTnC or to an epitope that is formed when the long CTnT is in complex with cTnI and cTnC or when cTnI and cTnC are in complex; and measuring the amount of standard cTnT in the sample by contacting the sample with a third and a fourth binder molecule specifically binding to different epitopes in a region corresponding to amino acids 69-189 of SEQ ID NO: 1; and
iii) calculating the ratio of long cTAT to standard cTnT in the sample on the basis of the measurements in step i) or i′) and ii).

The calculated ratio obtained in step iii) may then be used to determine, for example, whether the subject whose sample has been analysed for the ratio of long cTnT to standard cTnT is likely to have or not to have MI, turning also this method into an in vitro method of determining likelihood of MI in a subject. In some embodiments, the calculated ratio obtained in step iii) is compared to a corresponding reference ratio, preferably to a corresponding normal reference ratio for said determination.

It is to be understood that the above-disclosed method of calculating the ratio of long cTnT to standard cTnT can be converted into a method of calculating the ratio of standard cTnT to long cTnT by simply modifying step iii) such that the ratio of standard cTnT to long cTnT is calculated on the basis of the measurements in step i) or i′) and ii) instead of calculating the ratio of long cTnT to standard cTnT. As apparent to those skilled in the art, the previous paragraph applies also to the method disclosed in this paragraph.

In some embodiments, measuring the amount of standard cTnT may be carried out using a commercially available conventional hs-cTnT assay.

In some embodiments, calculating the ratio of long cTnT to standard cTnT in a sample may be replaced with calculating the ratio of long cTAT to C-terminal cTnT. Therefore, also provided is an in vitro method of calculating the ratio of long cTnT and C-terminal cTnT in a sample, the method comprising:
i) measuring the amount of long cTnT in the sample;
ii) measuring the amount of C-terminal cTnT in the sample; and
iii) calculating the ratio of long cTnT to C-terminal cTnT or the ratio of C-terminal cTnT to long cTnT in the sample on the basis of the measurements in step i) and ii).

To be more precise, the method of calculating the ratio of long cTnT and C-terminal cTnT in a sample may comprise:
i) measuring the amount of long cTnT in the sample by contacting the sample with a first binder molecule specifically binding to a region of cTnT corresponding to amino acids 201-288 of SEQ ID NO:1, to cTnI, to cTnC, or to an epitope that is formed when the long cTnT is in complex with cTnI and cTnC or when cTnI and cTnC are in complex, and a second binder molecule specifically binding to a region of cTAT corresponding to amino acids 1-200 of SEQ ID NO: 1; or
i′) measuring the amount of long cTnT in a sample by contacting the sample with a first binder molecule specifically binding to a region of cTnT corresponding to amino acids 195-220 of SEQ ID NO:1, and with and a second binder molecule specifically binding to a region of cTnT corresponding to amino acids 1-194 of SEQ ID NO: 1 or to a region of cTnT corresponding to amino acids 221-288, to cTnI, to cTnC or to an epitope that is formed when the long cTnT is in complex with cTnI and cTnC or when cTnI and cTnC are in complex; and
ii) measuring the amount of C-terminal cTnT ii) in the sample by contacting the sample with a fifth binder molecule specifically binding to an epitope in a region corresponding to amino acids 223-288 of SEQ ID NO: 1, and with a sixth binder molecule specifically binding to a different epitope in a region corresponding to amino acids 223-288 of SEQ ID NO: 1, to cTnI, to cTnC, or to an epitope that is formed when the C-terminal cTnT is in complex with cTnI and cTnC or when cTnI and cTnC are in complex; and
iii) calculating the ratio of long cTnT to C-terminal cTnT or the ratio of C-terminal cTnT to long cTnT in the sample on the basis of the measurements in step i) or i′) and ii).

In some embodiments, the first and/or the sixth binding molecule is capable of binding specifically to cTnI comprising an amino acid sequence depicted in SEQ ID NO: 2, preferably to amino acids corresponding to amino acids 23-197 in SEQ ID NO: 2, to cTnC comprising an amino acid sequence depicted in SEQ ID NO: 3, or to an epitope that is formed when the cTnT form to be quantitated is in complex with cTnI and cTnC or when cTnI and cTnC are in complex.

Again, the calculated ratio obtained in step iii) may then be used to determine, for example, whether the subject whose sample has been analysed for the ratio of long cTnT to C-terminal cTnT is likely to have or not to have MI, turning also this method into an in vitro method of determining likelihood of MI in a subject.

Quantitating long cTnT, or calculating the ratio of long cTnT to standard cTnT or the ratio of long cTnT to C-terminal cTnT may provide substantial help in clinical decision making in choosing necessary followup tests and appropriate treatment procedures. In some implementations, the present method of determining a subject's likelihood of having MI may further include therapeutic intervention. Once a subject is identified as likely having acute MI, he/she may be subjected to, for instance, coronary angiography followed by revascularization when needed and administration of antithrombotic therapy.

Thus, also provided is a method of treating MI in a subject in need thereof, wherein the method first comprises determining the likelihood of a subject suspected of having MI actually having MI, and if said subject is determined or identified as likely having MI, then subjecting the subject to any appropriate treatment of MI, such as coronary revascularization and administration of antithrombotic treatments.

### Example 1. Exemplary Three Tracer Long cTnT Assay

### Methods

### Patients

In troponin fragmentation in myocardial injury (Tropo-Fragm) study (ClinicalTrials.gov Identifier: NCT04465591) we evaluated cTnT fragmentation in 46 patients with non-ST elevation MI (NSTEMI), 71 patients with ST elevation MI (STEMI) and 40 patients with ESRD on maintenance hemodialysis. Clinical characteristics of the study groups are described in Table 1. Coronary angiography was performed in 44 (95.7%) patients in the NSTEMI group, 36 (78.3%) patients were treated with percutaneous coronary intervention (PCI) and 6 (13%) patients with coronary bypass surgery. All of the patients in the STEMI group received coronary angiography and primary PCI. All participants provided written informed consent. The study complies with Declaration of Helsinki as revised in 2002 and the study protocol was approved by the Medical Ethics Committee of the Hospital District of Southwest Finland.

TABLE 1
Baseline Characteristics of the
Patients in the Study Groups.

| | ESRD | NSTEMI | STEMI | |
|---|---|---|---|---|
| | (n = 40) | (n = 46) | (n = 71) | p |
| Age median (IQR) | 72 (59-76) | 73 (66-77) | 65 (57-72) | 0.002 |
| Female | 17 (42.5) | 12 (26.1) | 15 (21.1) | 0.052 |
| Current smoking | 4 (10.0) | 8 (17.4) | 28 (40.6) | 0.001 |
| Hypertension | 39 (97.5) | 32 (69.6) | 27 (38.0) | <0.001 |
| Diabetes | 25 (62.5) | 16 (34.8) | 9 (12.7) | <0.001 |
| Dyslipidemia | 32 (80.0) | 26 (56.5) | 18 (25.4) | <0.001 |
| Prior coronary | 18 (45.0) | 23 (50.0) | 9 (12.7) | <0.001 |
| artery disease | | | | |
| Prior myocardial | 12 (30.0) | 8 (17.4) | 5 (7.0) | 0.006 |
| infarction | | | | |
| Heart failure | 25 (62.5) | 3 (6.5) | 1 (1.4) | <0.001 |
| Atrial fibrillation | 16 (40.0) | 7 (15.2) | 6 (8.5) | <0.001 |

Data presented as cases/number (percentage, or median (IQR), as indicated.
ESRD indicates end-stage renal disease; NSTEMI, non-ST elevation myocardial infarction; STEMI, ST elevation myocardial infarction, IQR = interquartile range

### Blood Sampling

In patients with MI, the blood samples were collected at the earliest possible opportunity after hospital admission. In ESRD patients, the blood samples were collected during a dialysis clinic visit before and after the hemodialysis. Certified laboratory services by Turku University Hospital (TYKSLAB) took care of the blood samples. The 5 ml Li-heparin tube was centrifuged 15 minutes at 2200 g immediately after arrival to the laboratory and then the plasma was separated and stored at −70° C. until analysis. The samples were defrosted at room temperature prior to analysis, mixed to ensure homogeneity and centrifuged.

### Laboratory Methods

Standard cTnT Assay. All plasma samples were analyzed with Elecsys Troponin T hs kit using Cobas 8000 system (e801 module) (Roche Diagnostics GmbH, Mannheim, Germany). The Elecsys Troponin T hs assay uses two monoclonal antibodies which specifically target the central part of the human cardiac troponin T. The amino acid residue (aar) numbering in this article is based on the amino acid sequence of the dominant isoform of troponin in adult heart, Troponin T isoform 6 also known as TnT3 (UniProtKB/Swiss-Prot entry P45379-6) 16. The capture antibody of the standard cTnT assay recognizes epitope at aar 136-147 and the tracer antibody conjugated with Tris(2,2′-bipyridyl)ruthenium (II)-complex (Ru(bpy)) recognizes the epitope at aar 125-130. Thus, the assay targets the middle part of the cTnT molecule detecting all longer and shorter cTnT molecules that contain this part. The assay uses electrochemiluminescence detection. Calibration of the Roche assay is based on the Elecsys Troponin T STAT assay, which is originally standardized against Enzymun-Test Troponin T method. The laboratory's reporting limit for the Roche assay was 5 ng/l.

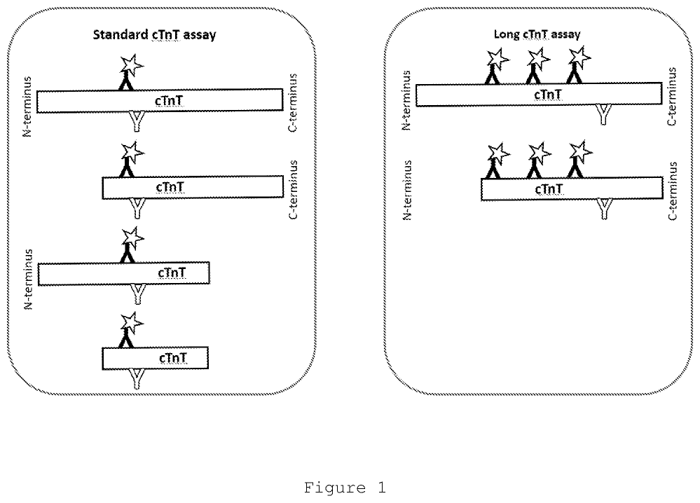

Long cTnT Assay Design. A novel sensitive time-resolved immunofluorometric assay was developed for the detection of long cTnT and the ratio of long cTnT and standard cTnT (cTnT ratio) was used a measure of troponin fragmentation. The long cTnT assay follows the sandwich type immunoassay format and utilizes time-resolved-fluorescence (TRF) as the measurement platform. The assay is based on the use of capture antibody targeting the C-terminal part of cTnT (mab 7E7, aar 223-242) and a combination of tracer antibodies targeting cTnT parts towards the N-terminus (mab 7G7, aar 67-86 in SEQ ID NO: 1; mab 329cc, aar 119-138 in SEQ ID NO: 1; mab 1C11cc, aar 171-190 in SEQ ID NO:1) (FIG. 1). The selected antibody combination allows the exclusion of most fragmented forms of cTnT and thus promotes the detection of the long cTnT molecules that span over multiple cleavage sites located at aar 190-233. The use of multiple tracer antibodies with separate epitopes allows enhancement of the signal from single cTnT molecules.

The monoclonal antibodies (mabs) for long cTnT detection were obtained from Hytest Ltd (Turku, Finland). Human cardiac troponin ITC-complex (Hytest Ltd, Turku, Finland) was used as a calibrator in the immunoassay as diluted in TSA-BSA buffer (tris-HCl [50 mmol/l] pH 7.75; NaCl [150 mmol/l]; NaN₃ [0.5 g/l]; bovine serum albumin [75 g/l, Probumin, Merck, Darmstadt, Germany]) and stored at −20° C. until use.

Antibody Conjugation with Biotin or Europium Chelate for Long cTnT Assay. The capture antibody (7E7) was conjugated with 30-fold molar excess of biotin isothiocyanate (Biotechnology University unit, of Turku, Turku, Finland) in a sodium carbonate-bicarbonate buffer (50 mmol/l; pH 9.8). The reaction mixture was kept at room temperature for 4 hours, after which the buffer was changed to TSA buffer [50 mmol/l] pH 7.75; NaCl [150 mmol/l]; NaN3 [0.5 g/l] with NAP-10 and PD-10 columns (GE Healthcare). The mab concentration of the collected eluate was measured with UV-VIS spectrophotometer at 280 nm. DTPA-purified BSA (Perkin Elmer) at 1 g/l was added to mab preparation, after which the solution was filtered through a 0.22 μm pore size filter and stored at 4° C.

The tracer antibodies (7G7, 329cc and 1C11cc) were individually conjugated with 30- to 35-fold molar excess of intrinsically fluorescent [2,2′,2″,2′″-{[2-(4-isothiocyanatophenyl)ethylimino]bis-(methylene)bis{4-{[4-(alfa-galactopyranoxy)phenyl]ethynyl}pyridine-6,2-diyl}bis(methylenenitrilo)}tetrakis(acetato)] europium(III) chelate17 (Biotechnology unit, University of Turku, Turku, Finland). The conjugation reactions were performed in sodium carbonate-bicarbonate buffer (50 mmol/l; pH 9.8) at room temperature overnight (18-20 h). The labelled antibody was purified with FPLC system equipped with Superdex 200 HR 10/30 gel filtration column and TSA buffer as an eluent. The fractions containing label-conjugated mab were identified with UV-detector and TRF-measurement (Arcus 1230, Wallac Oy). The selected fractions containing the labeled mabs were pooled and 1 g/l of DTPA-purified BSA was added, after which the solution was filtered through a 0.22 μm pore size filter and stored at 4° C.

Long cTnT Assay Protocol. Biotinylated capture antibody was added as 200 ng in 25 μl of Buffer Solution RED (Kaivogen) into streptavidin-coated microtiter wells (Kaivogen) and incubated for 1 h at room temperature. After incubation, the microtiter wells were washed with wash buffer (Kaivogen). The tracer antibodies (100 ng of each) were added to the wells in 40 μl of tracer buffer (Tris-HCl [100 mmol/l] pH 7.75; NaCl [600 mmol/l]; NaN3 [0.5 g/l]; BSA [25 g/l]; casein [4 g/l]; bovine-γ-globulin [0.6 g/l]; native mouse IgG [0.8 g/l]; denaturated mouse IgG [0.05 g/l]). Calibrator or sample (30 μL per well) was added to the wells as triplicates and the wells were covered with sealing tape and incubated for 1 h at +36° C., 900 rpm (iEMS incubator/shaker, Thermo Electron Corporation/Labsystems). The wells were then washed with the wash buffer and dried in a stream of hot air for 5 minutes. After the plate had cooled down to room temperature, the TRFsignal was measured (VictorX4, PerkinElmer) with excitation wavelength of 340 nm, emission wavelength of 615 nm, measurement delay of 250 μs, and measurement window of 750 μs. All analyses were performed as triplicates.

Analytical Performance of Long cTnT Assay. The analytical detection limit of the long cTnT assay (blank mean+3SD) was determined at 10.0 ng/L and the accuracy goal of 20% CV was met at 13.8 ng/L. The assay showed good linearity with STEMI plasma diluted in TSA-BSA buffer and proper recovery of ITC calibrator spiked into plasma samples.

### Statistical Analysis

Continuous variables were reported as mean+standard deviation (SD) and median (interquartile range [IQR]) for normally distributed and skewed variables, respectively. Normality in continuous covariates was analyzed with Shapiro-Wilk tests and Kolmogorov-Smirnov tests. The unpaired t test or one-way ANOVA and Mann-Whitney test or Kruskal-Wallis test were used to compare normally distributed and skewed continuous variables, respectively. In analyses between multiple subgroups Bonferroni correction was used to perform pairwise comparisons. Pearson's χ2 or Fisher's exact test were used to compare categorical covariates in the study subgroups. Categorical variables were reported with absolute and relative (percentage) frequencies. The association between continuous independent variables and the dependent variable was tested with linear regression models. The comparisons between the standard and long cTnT levels before and after hemodialysis in the ESRD group were performed using the Wilcoxon signed rank test.

Receiver operating characteristics (ROC) curve analyses were performed to estimate the area under the curve (AUC) to measure the discriminative capacity of long cTnT and cTnT ratio between ESRD and NSTEMI groups. All tests were two-sided, and significance was set at p<0.05. IBM SPSS Statistics software version 26.0 was used to perform all analyses.

### Results

Long cTnT, Standard cTnT and Long/Standard cTnT Ratio in STEMI, NSTEMI and ESRD Groups

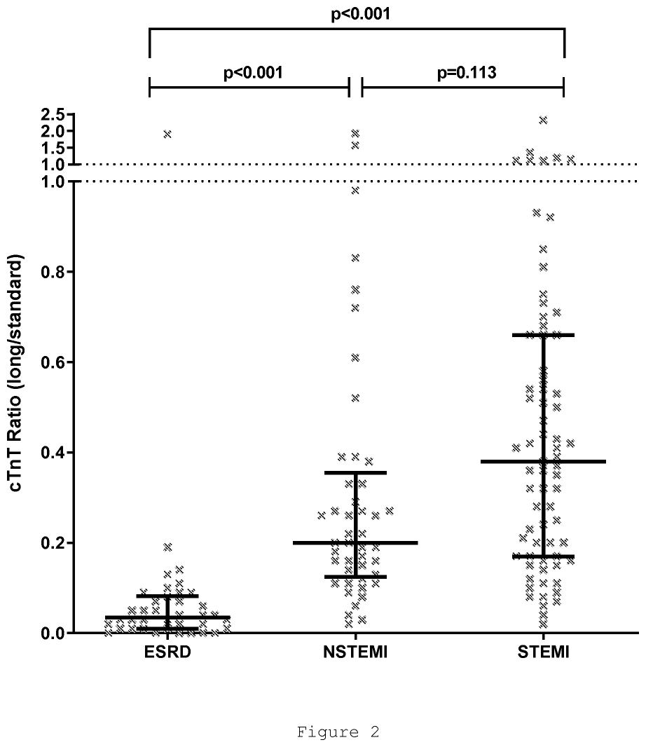

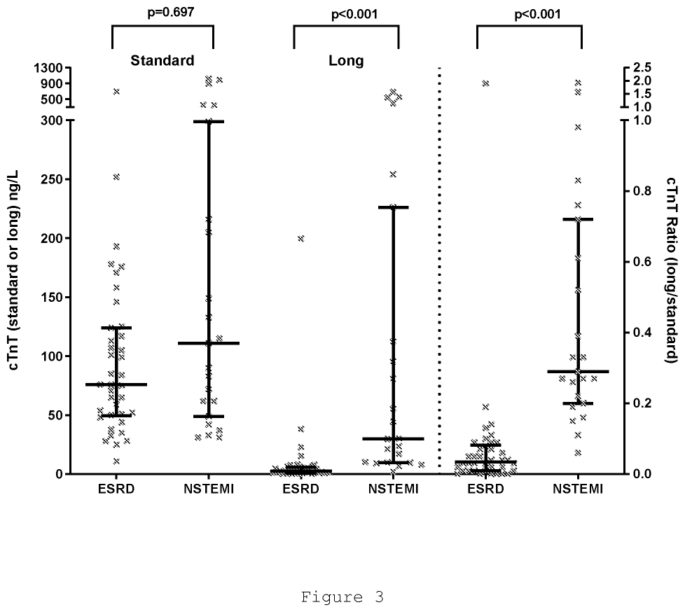

Patients with STEMI had the highest standard cTnT concentrations (Table 2). All but one patient with ESRD had standard cTnT values exceeding the uniform 99th percentile of 14 ng/L, but the standard cTnT concentrations did not differ significantly from the NSTEMI group (76 [50-124] ng/L vs. 141 [62-312] ng/L, p=0.063) (Table 2, FIG. 2). Long cTnT concentrations were lower in ESRD patients compared with NSTEMI and STEMI patients (3 [1-6] ng/L vs. 30 [10-186] ng/L vs. 442 [154-1235] ng/L, p<0.001 for both comparisons) (Table 2). Consequently, the ratio of long to standard cTnT was lower in in ESRD patients compared with both NSTEMI and STEMI patients (0.04 [0.01-0.08] vs. 0.20 [0.13-0.36] vs. 0.38 [0.17-0.66], p<0.001 for both comparisons) (Table 2, FIGS. 2 and 3).

TABLE 2
Renal Function, Standard and Long cTnT
and their Ratio in the Study Groups.

| | ESRD | | NSTEMI | | STEMI | | |
|---|---|---|---|---|---|---|---|
| | (n = 40) | | (n = 46) | | (n = 71) | | p |
| Creatinine μg/L | 570 | (441-743) | 79 | (69-100) | 76 | (65-86) | <0.001 |
| eGFR ml/min | 8 | (6-10) | 72 | (60-90) | 90 | (76-96) | <0.001 |
| Standard | 76 | (50-124) | 141 | (62-312) | 1471 | (565-4130) | <0.001 |
| cTnT ng/L | | | | | | | |
| Long cTnT ng/L | 3 | (1-6) | 30 | (10-186) | 442 | (154-1235) | <0.001 |
| cTnT ratio | 0.04 | (0.01-0.08) | 0.20 | (0.13-0.36) | 0.38 | (0.17-0.66) | <0.001 |
| (long/standard) | | | | | | | |

Data presented as median and interquartile range (IQR).
ESRD indicates end-stage renal disease; NSTEMI, non-ST elevation myocardial infarction; STEMI, ST elevation myocardial infarction; eGFR, estimated glomerular filtration range; cTnT, cardiac troponin T.

Changes in Long/Standard cTnT Ratio after MI

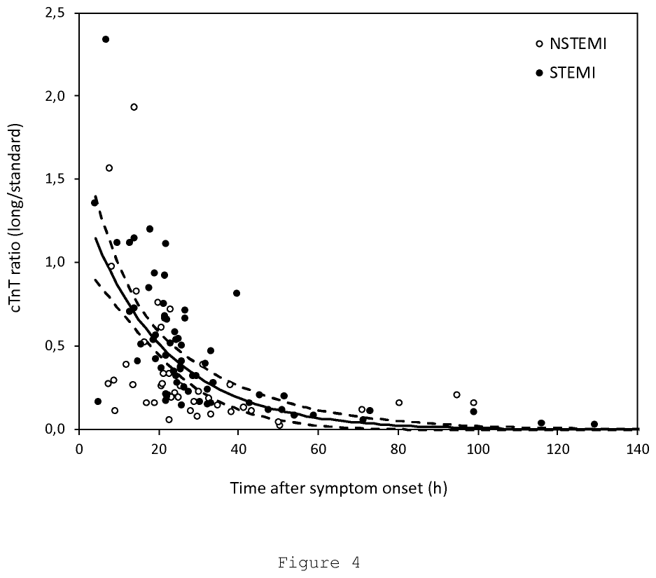

Delay between symptom onset and the study sample could be determined in 109 (93%) patients with MI. Long/standard cTnT ratio decreased in time with an average half-life of 13.9 hours. (FIG. 4). Consequently, patients with a ≤24-hour delay (n=23 (53.5%) and n=32 (48.5%) for NSTEMI and STEMI patients, respectively) had higher cTnT ratios compared to those with longer delays both in the NSTEMI (median 0.29 (0.20-0.72) [≤24h delay] vs. 0.15 (0.10-0.20) [>24h delay], p<0.001) and STEMI groups (median 0.62 (0.41-0.93) [≤24h delay] vs. 0.22 (0.11-0.38) [>24 h delay], p<0.001).

Long/Standard cTnT Ratio and Hemodialysis

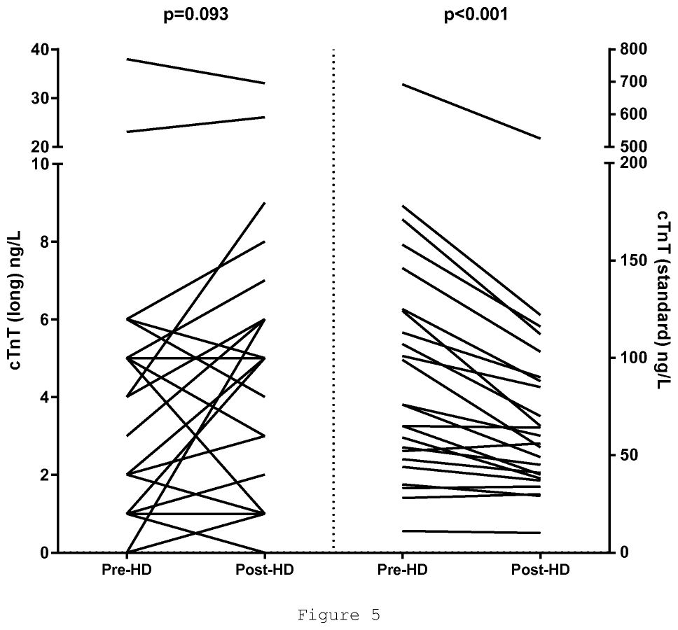

Baseline eGFR was not associated with the cTnT ratio in a univariate linear regression analysis (β 0.300, CI95% 0.0-0.013, p=0.053) in the NSTEMI group or in the STEMI group (β 0.045, CI95%−0.004-0.006, p=0.714). In ESRD patients, hemodialysis had no significant effect on long cTnT levels, but caused a minor decrease in standard cTnT concentrations and minor increase in long/standard cTnT ratio (0.04 vs 0.06, p=0.002) (FIG. 5).

Diagnostic Performance of Long/Standard cTnT Ratio

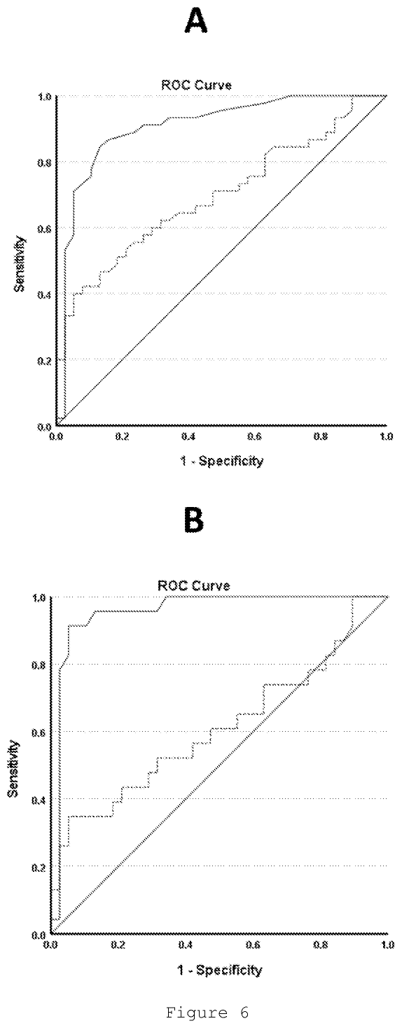

In the ROC curve analyses, cTnT ratio showed excellent predictive power in discriminating ESRD and NSTEMI patients with an AUC of 0.906 (CI95% 0.837-0.975) (all NSTEMI patients included) and 0.955 (CI95% 0.899-1.0) (only NSTEMI patients with a ≤24 h delay between symptom onset and study sample collection included). cTnT ratio was clearly superior to standard cTnT in discriminating between ESRD and NSTEMI (standard cTnT AUC 0.688 (CI95% 0.575-0.800) for all NSTEMI patients, standard cTnT AUC 0.609 (CI95% 0.454-0.764) for NTSEMI patients with <24 h delay between symptom onset and sample collection). The optimal cTnT ratio cutoff point of 0.105 showed a sensitivity of 84.4% and a specificity of 86.8% in the ROC analysis in separating ESRD patients and NSTEMI patients (all patients included). When only NSTEMI patients with a ≤24h delay between symptom onset and blood sample collection were included, the sensitivity was 91.3% and specificity 94.7% with the cutoff point of 0.145 (FIG. 6).

### Example 2. Exemplary One Tracer Long cTnT Assay

### Materials and Methods

Long cTnT Assay with One Tracer

The capture antibody (Hytest antibody mab 7E7, aar 223-242), that had been biotinylated as described in Example 1, was added as 200 ng in 25 μl of Buffer Solution RED (Kaivogen) into streptavidin-coated microtiter wells (Kaivogen) and incubated for 1 h at room temperature. After incubation, the microtiter wells were washed with wash buffer (Kaivogen). One tracer antibody (either Hytest antibody mab 7G7, aar 67-86; mab 329cc, aar 119-138; or mab 1C11cc, aar 171-190, labelled with intrinsically fluorescent europium (III) chelate as in Example 1) was added to the wells as 100 ng in 10 μl of tracer buffer (Tris-HCl [100 mmol/l] pH 7.75; NaCl [600 mmol/l]; NaN3 [0.5 g/l]; BSA [25 g/l]; casein [4 g/l]; bovine-γ-globulin [0.6 g/l]; native mouse IgG [0.8 g/l]; denaturated mouse IgG [0.05 g/l]). Calibrator or sample (30 μL per well) was added to the wells as triplicates and the wells were covered with sealing tape and incubated for 1 h at +36° C., 900 rpm (iEMS incubator/shaker, Thermo Electron Corporation/Labsystems). The wells were then washed with the wash buffer and dried in a stream of hot air for 5 minutes. After the plate had cooled down to room temperature, the TRF-signal was measured (VictorX4, PerkinElmer) with excitation wavelength of 340 nm, emission wavelength of 615 nm, measurement delay of 250 us, and measurement window of 750 us. All analyses were performed as triplicates.

Standard cTnT Assay

The samples were analysed with standard cTnT assay similarly as in Example 1

### Patients, Samples and Calibrators

A subset of the lithium heparin samples described in Example 1, collected from the patients described in Example 1, was included in this study. Similar calibrators as in Example 1 were used in this study.

### Statistical Analysis

Due to skewed distribution, continuous variables were reported as median (interquartile range [IQR]) and Mann-Whitney test was used for comparisons. IBM SPSS Statistics software version 26.0 was used to perform all analyses.

### Results

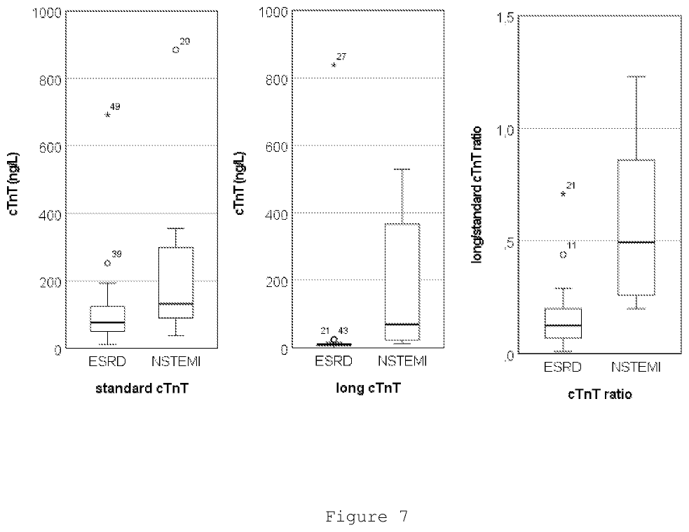

With the long cTnT assay that used only mab 329 as the tracer antibody, the concentration of long cTnT was significantly higher in samples collected from 10 consecutive NSTEMI patients within 24 hours after the symptom onset than in samples of 40 ESRD patients (69 ng/L [25-306 ng/L] vs. 10 ng/L [6-13 ng/L], p<0.001). The standard cTnT concentrations in the same groups were overlapping remarkably (132 ng/L [95-276 ng/L] vs. 76 ng/L [50-122 ng/L], p=0.450). Also the ratio of the long cTnT to standard cTnT was significantly higher in the same NSTEMI patients (0.50 [0.28-0.80] vs. 0.12 [0.07-0.20], p<0.001) (FIG. 7).

With the long cTnT assays that used either mab 7G7 or mab 1C11 as the single tracer antibody, similar high concentrations were measured in samples collected from STEMI patients as with the long cTnT assay that used mab 329 alone as the tracer or as with the assay of Example 1 that used the combination of antibodies mab 329, mab 1C11 and mab 7G7 as the tracer.

### Example 3. Exemplary Long cTnT Assays with Capture Antibodies Targeting cTnI or cTnI-TnC Complex

### Materials and Methods

Long cTnT Assays with Capture Antibodies Binding to cTnI or to cTnI-TnC Complex

cTnI-targeting capture antibodies (anti-cTnI mabs 19C7, aar 41-49, HyTest, and 817, aar 137-148, International Point of Care inc) and cTnI-InC complex targeting antibody (Tcom8, epitope is only present when cTnI and cTnC are in complex, Hytest) were biotinylated similarly as described for the capture antibody in Example 1. The antibodies were added (either 100 ng 19C7 and 100 ng 817 or 200 ng Tcom8) in 25 μl of Buffer Solution RED (Kaivogen) into streptavidin-coated wells and incubated for 1 h at room temperature. After incubation, the microtiter wells were washed with wash (Kaivogen). A combination of cTnT-targeting buffer tracer antibodies (Hytest antibodies mab 7G7, aar 67-86; mab 329cc, aar 119-138; and mab 1C11cc, aar 171-190, labelled with intrinsically fluorescent europium (III) chelate as in Example 1) were added to the wells as 100 ng of each in 40 μl of tracer buffer (Tris-HCl [100 mmol/l] pH 7.75; NaCl [600 mmol/l]; NaN3 [0.5 g/l]; BSA [25 g/l]; bovine-γ-globulin [0.6 g/l]; native mouse IgG [0.8 g/l]; denaturated mouse IgG [0.05 g/l]; casein [4 g/l]). Calibrator or sample (30 μL per well) was added to the wells as triplicates and the wells were covered with sealing tape and incubated for 1 h at +36° C., 900 rpm (iEMS incubator/shaker, Thermo Electron Corporation/Labsystems). The wells were then washed with the wash buffer six times and dried in a stream of hot air for 5 minutes. After the plate had cooled down to room temperature, the TRF-signal was measured (Victorx4, PerkinElmer) with excitation wavelength of 340 nm, emission wavelength of 615 nm, measurement delay of 250 μs, and measurement window of 750 μs.

### Other Assays

The samples were analysed with the standard cTnT assay, i.e. commercial Roche hs-cTnT assay, similarly as in Example 1. The samples were analysed also with the long cTnT assay described in Example 1 in which the capture antibody is anti-cTnT mab 7E7 (Hytest).

### Patients, Samples and Calibrators

A small subset of the lithium heparin plasma samples described in Example 1, collected from the patients described in Example 1, was included in this study. Similar calibrators (troponin I-T-C complex from Hytest) as in Example 1 were used in this study.

### Results

All assays (standard cTnT assay, long cTnT assay with anti-cTnT capture mab, long cTnT assay with anti-cTnI capture mabs and long cTnT assay with anti cTnI-InC complex capture mab) gave highly elevated results with STEMI patients (Table 1). While the standard cTnT assay gave highly elevated results also for ESRD patients, the other assays did not detect anything or only negligible amounts in the ESRD samples (Table 1).

The differing values detected by the different long cTnT for STEMI patients may originate from differences in the fractions of cTn complexes present in endogenous samples and in the calibration material used in the study (e.g. lesser amount of complexed cTn in the calibration material possibly due to instability would lead to increased result for endogenous samples with assays utilizing complex-dependent epitopes.) However, the results show that discrimination between MI and ESRD patients can be achieved with each described long cTnT assay setup.

TABLE 3
Samples of STEMI and ESRD patients analysed
with long cTnT assays with different setups.

| | Roche | Long cTnT assay | Long cTnT assay | Long cTnT assay with |
|---|---|---|---|---|
| | cTnT | with anti-cTnT | with anti-cTnl | anti-cTnl/TnC |
| | assay | capture mab | capture mabs | capture mab |
| | (ng/L) | (ng/L) | (ng/L) | (ng/L) |
| STEMI A | 1403 | 952 | 4513 | 519 |
| STEMI B | 2462 | 1844 | 14966 | 3742 |
| ESRD A | 692 | 4 | 1 | 0 |
| ESRD B | 193 | 1 | 0 | 0 |

### Example 4. Exemplary Long cTnT Assays with Upconverting Nanoparticles as Labels

### Materials and Methods

Conjugation of Upconverting Nanoparticles with Antibodies

Oleic acid-capped NaYF₄: 17% Yb³⁺, 3% Er³⁺ upconverting nanoparticles (UCNP) (University of Turku, Finland) were coated with poly (acrylic acid) (PAA) and conjugated with monoclonal anti-cTnT tracer antibodies (Hytest) as described before (Raiko et al., Clin Chim Acta. 2021 December; 523:380-385) using 2.5 mM EDC (mab 1C11, aar 171-190) or 30 mM EDC (mab 329cc, aar 119-138, and mab 7G7, aar 67-86) for conjugation.

Long cTnT Assays with Upconverting Nanoparticles

Anti-cTnT antibody 7E7 (aar 223-242, HyTest) used as capture antibody was biotinylated as in Example 1. The tracer dilution containing 4 ng/μl of one UCNP-conjugated tracer antibody (either mab 329cc, mab 1C11 or mab 7G7) was prepared 30 min before starting the assay using assay buffer (Kaivogen) supplemented with 1 mM KF, 0.05% PAA, 0.2% fat-free milk powder, 0.08% native mouse IgG, and 0.005% denatured mouse IgG. The capture antibody was added as 200 ng in 50 μl of assay buffer into C8 Lockwell LUMI White Maxisorp microtiter plate (Thermo Scientific) wells that had been passively coated with 1 μg/well streptavidin (Biospa) as described previously (Raiko et al., Clin Chim Acta. 2021 December; 523:380-385). After incubation of 30 min at room temperature in slow shaking the wells were washed with wash buffer (Kaivogen).

The calibrators and samples were diluted in sample buffer (37.5 mM Tris pH 8, 500 mM NaCl, 0.06% bovine γ-globulin, 2.5% BSA, 5% D-trehalose, 37.5 U/ml heparin, 0.08% native mouse IgG, 0.005% denatured mouse IgG, 0.2% casein, 0.0375% NaN3) in a 4:1 sample-to-buffer ratio. The volume of 50 μl of sample or calibrator was added to the wells. After incubation of 30 min at room temperature in slow shaking the wells were washed with the wash buffer.

The tracer dilution was subjected to sonication and 50 μl of the dilution was added to each well. After incubation of 15 min at room temperature in slow shaking the wells were washed with the wash buffer and the wells were allowed to dry for 1.5 h before signal measurement. The upconversion luminescence was measured from the bottom of the wells at 540 m, using a modified Plate Chameleon microplate reader (Hidex) equipped with a 980 nm laser for excitation.

### Patients, Samples and Calibrators

Two STEMI patients that were included in Example 1 were included also in this study. Lithium heparin plasma samples and serum samples were collected from the patients at the same time. Similar calibrators (troponin I-T-C complex from Hytest) as in Example 1 were used in this study.

### Results

All assays produced highly elevated results with heparin plasma samples of both patients (mean 420 ng/L, SD 141 ng/L). However, with serum samples only the assays with 1C11 or 329cc as the tracer antibody produced highly elevated results representing on average 75% (SD 3%) of the levels detected with the same assays in heparin plasma samples. With tracer antibody 7G7 the results of serum samples were remarkably lower than with the heparin plasma samples. The concentrations that were detected in serum with 7G7 tracer antibody were only 6% for patient 1 and 8% for patient 2 compared to the concentrations detected with heparin plasma samples with the same assay.

The epitope of antibody 7G7 lies at aar 67-86. There is a prominent cleavage site of cTnT between aar 68 and 69. Thrombin is known to cleave cTnT between aar 68 and 69. As coagulation pathways become activated when serum samples are prepared, also thrombin becomes activated when serum samples are produced. Thus, in serum samples cTnT has been cleaved between aar 68 and 69 and the epitope of antibody 7G7 has been broken. As a result, we see low concentrations of long cTnT in serum samples that utilize mab 7G7 as the tracer antibody. This effect is not seen with the tracer antibodies 1C11 and 329cc which have their epitopes closer to the C-terminal end of cTnT.

The results emphasize that a vast majority of the cTnT in the circulation of MI patients is in a form that is not truncated from the N-terminal end and thus it is highly essential to measure also the intact form of cTnT.

It is obvious to a person skilled in the art that with the advancement of technology, the basic idea of the invention may be implemented in various ways. The invention and its embodiments are thus not limited to the examples described above, instead they may vary within the scope of the claims.

1. An in vitro assay method of determining likelihood of acute myocardial infarction (MI) in a subject, the method comprising:
i) measuring the amount of a first set of cardiac troponin (cTnT) molecules in a sample obtained from the subject, the first set of cTnT molecules consisting of intact cTnT and C-terminal cTnT forms that are not cleaved at a region corresponding to amino acids 189-223 of SEQ ID NO: 1 and comprise a region corresponding to amino acids 205-215 SEQ ID NO: 1; and
ii) determining the likelihood of acute MI in the subject on the basis of the measurement.

2. The in vitro assay method according to claim 1, wherein the amount of the first set of cTnT molecules in the sample is measured using a first binder molecule specifically binding to a region of cTnT corresponding to amino acids 201-288 of SEQ ID NO: 1, to cardiac troponin I (cTnI), to cardiac troponin C (cTnC), or to an epitope that is formed when the first set of cTnT molecules is in complex with cTnI and cTnC or when cTnI and cTnC are in complex and a second binder molecule specifically binding to a region of cTnT corresponding to amino acids 1-200 of SEQ ID NO:1.

3. The in vitro assay method according to claim 2, wherein the first binder molecule is a capture molecule immobilized on a solid surface and the second binder molecule is a tracer molecule containing a detectable label.

4. The in vitro assay method according to claim 3, wherein the capture molecule binds specifically to an epitope of cTnT corresponding to amino acids 223-242 of SEQ ID NO: 1.

5. The in vitro assay method according to claim 3, wherein one, two or three tracer molecules are used, each binding specifically to a different epitope of cTnT selected from epitopes corresponding to amino acids 67-86, 119-138 or 171-190 of SEQ ID NO: 1.

6. The in vitro assay method according to claim 1, wherein the amount of the first set of cTnT molecules is determined by a competitive assay, and the amount of the first set of cTnT molecules in the sample is measured using a binder molecule specifically binding to a region of cTnT corresponding to amino acids 190-223, preferably amino acids 195-220 of SEQ ID NO: 1.

7. The in vitro assay method according to claim 1, wherein the amount of the first set of cTnT molecules in the sample is measured using a first binder molecule specifically binding to a region of cTnT corresponding to amino acids 195-220 of SEQ ID NO:1, and a second binder molecule specifically binding to a region of cTnT corresponding to amino acids 1-194 of SEQ ID NO:1, to a region of cTnT corresponding to amino acids 221-288, to cTnI, to cTnC, to an epitope that is formed when the C-terminal cTnT is in complex with cTnI and cTnC, or to an epitope that is formed when cTnI and cTnC are in complex.

8. The in vitro assay method according to claim 7, wherein the first binder molecule is a capture molecule immobilized on a solid support, or the second binder molecule is a tracer molecule containing a detectable label, or wherein the first binder molecule is a tracer molecule containing a detectable label, and the second binder molecule is a capture molecule immobilized on a solid support.

9. The in vitro assay according to claim 1, further comprising:
measuring the amount of a second set of cTnT molecules in the sample, the second set of cTnT molecules consisting of all cTnT molecules in the sample comprising a central region of cTnT; or
measuring the amount of a third set of cTnT molecules in the sample, the third set of cTnT molecules consisting of all cTnT molecules in the sample comprising a C-terminal region of cTnT; and
calculating the ratio of the first set of cTnT molecules to the second set of cTnT molecules, the ratio of the second set of cTnT molecules to the first set of cTnT molecules, the ratio of the first set of cTnT molecules to the third set of cTnT molecules, or the ratio of the third set of cTnT molecules to the first set of cTnT molecules; and
determining the likelihood of acute MI in the subject on the basis of the calculated ratio.

10. The in vitro assay method according to claim 9, wherein the amount of the second set of cTnT molecules in the sample is measured using a third and a fourth binder molecule specifically binding to different epitopes in a region corresponding to amino acids 69-189 of SEQ ID NO: 1.

11. The in vitro assay method according to claim 10, wherein the third binder molecule specifically binds to an epitope corresponding to amino acids 136-147 of SEQ ID NO:1, the third binder molecule being preferably a capture molecule immobilised on a solid surface.

12. The in vitro assay method according to claim 10, wherein the fourth binder molecule specifically binds to an epitope corresponding to amino acids 125-130 of SEQ ID NO:1, the fourth binder molecule being preferably a tracer molecule comprising a detectable label.

13. The in vitro assay method according to claim 9, wherein the amount of the third set of cTnT molecules in the sample is measured using a fifth and a sixth binder molecule specifically binding to different epitopes in a region corresponding to amino acids 223-288 of SEQ ID NO: 1.

14. The in vitro assay method according to claim 9, wherein the amount of the third set of cTnT molecules in the sample is measured using a fifth binder molecule specifically binding to an epitope in a region corresponding to amino acids 223-288 of SEQ ID NO: 1, and a sixth binder molecule specifically binding to cTnI cTnC, or to an epitope that is formed when the third set of cTnT molecules is in complex with cTnI and cTnC or when cTnI and cTnC are in complex.

15. The in vitro assay method according to claim 1, for use in excluding the likelihood of the subject having MI; confirming the likelihood of the subject having MI; distinguishing patients having MI from patients showing elevated standard cTnT levels for a reason other than MI.

16. The in vitro assay method according to claim 1, wherein the sample is a blood sample, a serum sample or a plasma sample.

17. A kit for use in determining likelihood of MI in a subject, the kit comprising:
i) a first binder molecule capable of specifically binding to a region of cTnT corresponding to amino acids 201-288 of SEQ ID NO:1, to cTnI comprising an amino acid sequence depicted in SEQ ID NO: 2, to cardiac troponin C (cTnC) comprising an amino acid sequence depicted in SEQ ID NO: 3, or to an epitope that is formed when the first set of cTnT molecules is in complex with cTnI and cTnC or when cTnI and cTnC are in complex, and a second binder molecule capable of specifically binding to a region of cTnT corresponding to amino acids 1-200 of SEQ ID NO:1; or
i′) a first binder molecule specifically binding to a region of cTnT corresponding to amino acids 195-220 of SEQ ID NO:1, and a second binder molecule specifically binding to a region of cTnT corresponding to amino acids 1-194 of SEQ ID NO:1 or to a region of cTnT corresponding to amino acids 221-288, to cTnI, to cTnC, to an epitope that is formed when the C-terminal cTnT is in complex with cTnI and cTnC, or to an epitope that is formed when cTnI and cTnC are in complex; or
ii) a seventh binder molecule capable of specifically binding to a region of cTnT corresponding to amino acids 190-223, preferably 195-220 of SEQ ID NO: 1 and detectably labelled cTnT or a peptide thereof having at least partly intact C-terminal cleavage area.

18. The kit according to claim 17, further comprising a third and a fourth binder molecule capable of specifically binding to different epitopes in a region corresponding to amino acids 69-189 of SEQ ID NO: 1.

19. The kit according to claim 17, further comprising i) a fifth and a sixth binder molecule capable of specifically binding to different epitopes in a region corresponding to amino acids 223-288 of SEQ ID NO:1, or ii) a fifth binder molecule capable of specifically binding to an epitope in a region corresponding to amino acids 223-288 of SEQ ID NO:1, and a sixth binder molecule specifically binding to cTnI, to cTnC or to an epitope that is formed when the cTnT to be measured is in complex with cTnI and cTnC.

20. The kit according to claim 19, wherein the first binder molecule (if present), the third binder molecule (if present), the fifth binder molecule (if present) and the seventh binder molecule (if present) are capture molecules immobilised on a solid surface, or
wherein the second binder molecule, the fourth binder molecule (if present), the sixth binder molecule (if present) are tracer molecules comprising a detectable label; or
wherein the first binder molecule (if present), the third binder molecule (if present), the fifth binder molecule (if present) and the seventh binder molecule (if present) are tracer molecules comprising a detectable label; or
wherein the second binder molecule, the fourth binder molecule (if present), the sixth binder molecule (if present) are capture molecules immobilized on a solid surface.

21.-25. (canceled)
